(19) United States
(12) Patent Application Publication
(10) Pub. No.: US 2021/0222166 A1
(43) Pub. Date: Jul. 22, 2021

(54) LNA BASED NANODEVICE

(71) Applicant: AARHUS UNIVERSITET, Aarhus C (DK)

(72) Inventors: Jesper Sejrup Nielsen, Randers SØ (DK); Jørgen Kjems, Risskov (DK); Veronica Liv Andersen, Tranbjerg J (DK)

(21) Appl. No.: 16/625,915

(22) PCT Filed: Jul. 3, 2018
(86) PCT No.: PCT/EP2018/067886

§ 371 Date: Dec. 23, 2019

(30) Foreign Application Priority Data
Jul. 7, 2017 (EP) 17180234.1

(51) Int. Cl. C12N 15/113

(52) U.S. Cl. CPC C12N 15/113; C12N 2320/51; C12N 2310/3231; C12N 2310/3515; C12N 2310/53; C12N 2320/32; C12N 2310/3519; C12N 2310/315; C12N 2310/321

(57) ABSTRACT

The present invention relates to a nanodevice comprising of a branched nucleic acid structure comprising at least three double-stranded arms, wherein at least two of said double-stranded arms comprise LNA and further comprising DNA and/or modified RNA, wherein at least one of said double-stranded arms is modified with at least one functional chemical group, at least one linker and/or at least one ligand.

### TECHNICAL FIELD

The present invention relates to a nanodevice comprising of a branched nucleic acid structure comprising at least three double-stranded arms, wherein at least two of said double-stranded arms comprise LNA and further comprising DNA and/or modified RNA, wherein at least one of said double-stranded arms is modified with at least one functional chemical group, at least one linker and/or at least one ligand.

This application incorporates by reference a Sequence Listing with this application as an ASCII text file entitled “Sequence list_ST25_corrected.txt” created on Jan. 5, 2021 having a size of 4,474 bytes.

### BACKGROUND

Traditional medicine is made up of small, chemically synthesized molecules that are administered either by ingestion or by intravenous, intramuscular or subcutaneous injection. Such systemic treatments are often subject to adverse effects because the drug affects healthy, as well as diseased tissue. As a result, many elderly and immunocompromised patients are often excluded from certain types of treatments (in particular chemotherapy) because the side effects outweigh the potential benefit of the drug. Drug development companies have therefore invested heavily in identifying new classes of therapeutics with higher specificity and/or selectivity. When the molecular mechanism and ubiquitous nature of RNA interference were first uncovered, it was believed to revolutionize therapeutics as we know it^{[1]}. However, its translation from the bench to the clinic has been uncharacteristically slow and paved with complications. The major obstacle has been the lack of a safe and efficient means of delivery.

Recent advances in nanotechnology have enabled the development of several liposomal and polymeric systems which condense the payload into nanoparticles^{[2]}. Such nanoparticles offer superior pharmacokinetics over traditional drugs which often translate into higher therapeutic effects and lower toxicity. However, current nanoparticles suffer from a number of drawbacks including reduced biocompatibility and complicated and inefficient synthesis. Moreover, most nanoparticles are heterogeneous in size and surface properties and do not enter specific cells without extensive modification. Consequently, intracellular targets are often inaccessible which effectively eliminates an entire class of potential drugs from the pipeline. Efforts to improve the versatility of existing drug delivery platforms by introducing functionalities such as targeting or bioresponsiveness^{[3]} have showed only moderate success and do not allow combination of diagnostic and therapeutic options within the same system. Thus, there is an unmet need for a nanodevice having a high stability especially in biological fluids, which is easy and cost effective to synthesize even when conjugated with different ligands.

### SUMMARY

The present invention provides a nucleotide based nanodevice having a high stability in biological fluids, such as for example blood. Furthermore, the nanodevice of the present invention is easy to synthesize, the assembly process is extremely robust and it can be produced at low costs. The nanodevice of the present invention can be easily attached to for example targeting agents, drugs, imaging agents, antibodies and/or small molecules making it especially suitable for e.g. targeted drug delivery and bioimaging.

Accordingly, one aspect of the present invention relates to a nanodevice comprising of a branched nucleic acid structure comprising at least three double-stranded arms, wherein at least two of said double-stranded arms comprise:
LNA and/or modified LNA; and
at least one component selected from the group consisting of DNA, Phosphorothioate-DNA, morpholino-DNA, 2′-O-MeRNA, 2′-F-RNA, 2′MOE-RNA, 2′-O-Me-Phosphorothioate-RNA, 2′-F-Phosphorothioate-RNA, 2′MOE-Phosphorothioate-RNA, 2′-O-morpholino-MeRNA, 2′-F-morpholino-RNA and 2′MOE-morpholino-RNA,

wherein at least one of said double-stranded arms is modified with at least one functional chemical group, at least one linker and/or at least one ligand.

In one embodiment thereof the double-stranded arms comprise:
LNA and/or modified LNA; and
at least one component selected from the group consisting of DNA, 2′-O-MeRNA, 2′-F-RNA and 2′MOE-RNA.

A preferred embodiment of the present invention relates to a nanodevice comprising of a branched nucleic acid structure comprising at least three double-stranded arms, wherein at least two of said double-stranded arms comprise locked nucleic acid (LNA) and 2′-OMe-RNA and wherein at least one of said double-stranded arms is modified with at least one functional chemical group, at least one linker and/or at least one ligand.

The nanodevice of the present invention comprises at least three nucleotide strands.

In one embodiment said nanodevice comprises 3 to 6 double-stranded arms. Preferably, said nanodevice comprises 3 to 5 double-stranded arms. More preferably, said nanodevice comprises four double-stranded arms.

In one embodiment at least one of said nucleotide strands comprises LNA nucleotides and at least one of said nucleotide strands comprises of 2′-OMe-RNA nucleotides. The nucleotide strands preferably have a length of from 6 to 20 nucleotides.

In one embodiment said nanodevice comprises at least one 2′-amino-LNA. In one embodiment thereof, said functional chemical group, linker and/or ligand is attached to the amino-group of said 2′-amino-LNA.

The nanodevice of the present invention may for example comprise at least one ligand and further comprises at least one functional chemical group and/or at least one linker. In one embodiment said ligand is attached to the functional chemical group or the linker. In one embodiment thereof, said ligand is attached to the linker and wherein the linker is attached to the functional chemical group.

In one embodiment said nanodevice comprises at least two ligands, such as at least three ligands or such as at least four ligands. The nanodevice may for example comprise at least two different ligands, such as at least three different ligands or such as at least four different ligands.

In one embodiment said ligand(s) is/are selected from the group consisting of therapeutic agents, imaging agents, targeting agents, aptamers, vitamins, antibodies, peptides, albumin, oligonucleotides, fluorophores, lipids, tags, small molecules and reactive chemical groups.

In one embodiment said functional chemical group is selected from the group consisting of amines, amides, thiols, phosphates, carboxylates, haloacetyls, azides and aldehydes.

In one embodiment said linker is selected from the group consisting of N-Hydroxysuccinimide, maleimide and dibenzocyclooctyne.

Another aspect of the present invention relates to a nanodevice as defined herein for use as a medicament. In one embodiment thereof said nanodevice comprises a drug.

Yet another aspect of the present invention relates to a pharmaceutical composition comprising the nanodevice as defined herein. Preferably, the pharmaceutical further comprises a pharmaceutically acceptable carrier.

A further aspect of the present invention relates to a method of treating, preventing or ameliorating a disease by administering to a subject in need thereof a therapeutically effective amount of the pharmaceutical composition described herein. Preferably, said subject is a human.

In one aspect the present invention relates to use of a nanodevice as defined herein for bioimaging.

In another aspect, the present invention relates to use of a nanodevice as defined herein for drug delivery. Preferably, said drug delivery is targeted drug delivery.

### DESCRIPTION OF DRAWINGS

FIG. 1. (A) Schematic illustration of the Holliday junction. Each strand consists of 8 2′OMe nucleotides and 4 LNAs. LNAs are indicated by a grey dot. (B) Native polyacrylamide gel showing the assembly of the Holliday junction. (C) Melting curve of Holliday junction scaffold based on SYBR Gold binding. The apparent Tₘ is 82.3° C.

FIG. 2. Serum stability of HJ. 10 pmol of each HJ module were assembled in TAEM. At time 0, 10% FBS was added. 1 μl samples were withdrawn at the indicated timepoints and stored at −20° C. until the end of the experiment. As a control, we used a small double-stranded RNA which is quickly degraded.

FIG. 3. Assembly of functionalized HJ. Each functionalized oligo was purified by HPLC as described. Structures were assembled by mixing equimolar amounts of each of the four oligos in TAEM. (A) HJs with 1 and 2 folate. (B) HJs with 1-4 DOTA. (C) HJs with 1-3 triGalNAc. (D) HJs with A10 and GL21 aptamer. (E) HJs with one or more PEG. (F) HJs with αSynuclein. (G) HJs with THR and Penetratin peptide. (H) HJs with 1-3 biotin with or without streptavidin added.

FIG. 4. HJ and PEGylated HJs only induce a mild TNF-α response in monocyte culture supernatant.

FIG. 5. Blood circulation time and biodistribution of umodified HJ after I.V. or S.C. injections. Samples were withdrawn at the indicated timpoints and quantified. After 24 hrs, the animals were sacrificed and the organs scanned. The organs showed are from left to righ: liver, kidney, spleen, heart, lung. Left upper panel show blood levels of fluorescent HJs at different timepoints following IV or SC injection. The normalized fluorescent signal is shown in the graph to the right.

FIG. 6. Blood circulation time and biodistribution of non-functionalized and PEGylated HJs. Normalized blood levels are shown in the graph to the left whereas whole body images and organs after 24 hrs are shown to the right.

FIG. 7. Specific uptake of Cy5 labeled HJs in HepG2 and KB cells using triGalNAc and folate as targeting agents respectively. Uptake was quantified by flow cytometry.

FIG. 8. TriGaINAc targets HJs specifically to the liver. (A) biodistribution of HJs 24 hrs. post injection. (B) Time-course accumulation of HJs in the liver of HJ and HJ-2xTriGaINAc immediately after injection. (C) Quantification of data from (B).

FIG. 9. Targeting PSMA-positive cells in vitro and in vivo. (A) Illustration of the HJ-A10 construct. (B) Confocal microscopy showing specific uptake of HJ-A10. (C) Flow cytometry data of HJ and HJA10 uptake in LNCaP or PC3 cells. (D) Specific targeting of LNCaP tumors by HJ-PEG-A10.

FIG. 10

Upper Part:

PSMA specific aptamer (a9g) was conjugated to HJ oligos via an azido-DBCO reaction. HJs were assembled with 1, 2 or 3 aptamers as well as a Cy5 label.

Assembly was monitored by gel electrophoresis

Lower Part:

The labeled HJs were incubated with PSMA positive (PC3+) and PSMA negative (PC3−) cells for 45 min. Cellular uptake was monitored by flow cytometry and confocal microscopy. As a negative control, we used a PSMA unspecific aptamer (GL21) which is only slightly taken up by PC3 cells. Flow cytometry data was normalized to naked HJ in each cell line.

FIG. 11

Upper Part:

Assembly of HJ with up to three TfRL peptides as monitored by gel electrophoresis followed by SybrGold staining.

Lower Part:

Structure of the TfRL peptide. Cellular uptake (KB cells) of Cy5 labled HJ with or without TfrL as determined by flow cytometry.

FIG. 12

Upper Part:

Assembly of HJ with Her2 specific nanobody (Rb17c). The nanobody was conjugated to HJ oligos via a maleimide-thiol reaction. HJs were assembled with 1, 2 or 3 nanobodies as well as a Cy5 label. Assembly was monitored by gel electrophoresis

Lower Part:

The Cy5-labeled HJs were incubated with Her2 positive cells (SKBR3) and cellular binding subsequently monitored by flow cytometry and confocal microscopy. Flow cytometry data was normalized to naked HJ.

FIG. 13

Upper Part:

Two palmitoyl groups were added to the 3′end of Q3 via two aminoLNA-Palmitoyl T. Cy5.5 labeled HJs were assembled with or without the palmitoyls and injected intravenously in mice. Blood samples were drawn at different timepoints. The graph shows a plot of percent remaining HJ vs. time. N=5

Lower Part:

After 24 hrs, the mice were sacrificed and their organs collected and scanned. A representative image comparing a naked HJ to one containing the two palmitoyls is shown on the right. Organs are (from left to right) kidneys, spleen, liver). On the left is shown the biodistribution in the different organs (average of at least 5 animals).

FIG. 14

Schematic illustration of a nucleic acid barcode system for the HJ platform. HJs with different configurations of targeting agents, cargo, PK enhancers etc are assembled individually. Each configuration is identified by a unique barcode sequence which could be a short (or longer) oligoncleotide extension. The different HJs are then mixed together and screened in an appropriate model (i.e. a cell line, an animal, an immobilized ligand etc.). At the end of the screen, the optimal configuration can be identified via its unique barcode.

FIG. 15.

Three alternative ways of attaching unique barcodes. Barcodes can be attached as required via a click reaction similar to the attachment of targeting agents and fluorophores. A barcode can also be attached directly during synthesis of the individual HJ modules. A unique barcode could also be attahced by hybridising to a constant overhang build into one of the original HJ modules.

FIG. 16.

Example of a barcode system (proof of principle). A DNA barcode containing two M13 primer binding sites is clicked onto one of the HJ strands via an azido-DBXO reaction.

The barcode can then be quantified (read) by standard probe based qPCR. As proof of principle, we attached two barcode sequences. On the right side is a standard curve for a dilution series of each barcode sequence (Bar1 and Bar2). Below is shown the result of multiplex quantification of Bar1 (SEQ ID NO: 12) and Bar2 (SEQ ID NO: 13) mixed in different stoichiometries. The results show good correlation between expected and measured amounts across different ratios.

### DETAILED DESCRIPTION

Definitions

The term “nucleotide” as used herein defines a monomer of RNA or DNA. A nucleotide is a nucleobase to which a phosphate group is attached through a ribose or a deoxyribose ring. Mono-, di-, and tri-phosphate nucleosides are referred to as nucleotides.

The term “locked nucleic acid” or “LNA” as used herein refers to a modified RNA nucleotide, wherein the ribose moiety is modified with an extra bridge connecting the 2′ oxygen and 4′ carbon. The bridge “locks” the ribose in the 3′-endo (North) conformation, which is often found in the A-form duplexes. LNA nucleotides can hybridise with DNA or RNA according to Watson-Crick base-pairing rules. The locked ribose conformation enhances base stacking and backbone pre-organization and results in increased stability of the double-stranded oligonucleotide. The LNA can be either alpha or beta configuration such as beta-D-LNA (Formula I) or alpha-D-LNA (Formula II).

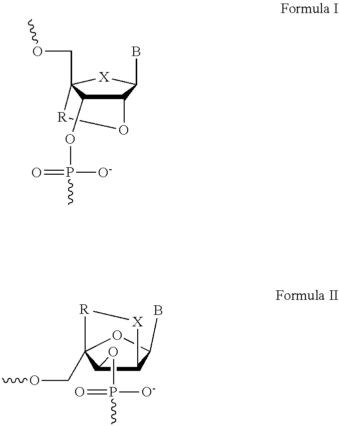

In formulas I and II R represents (CH₂)ₙ, where n is 1, 2 or 3. X represents O, S or N—R′, where R′ represents H or C₁₋₆-alk(en/yn)yl. Preferably, X is O or N—R′. More preferably X is O.

The term “C₁₋₆-alk(en/yn)yl” means C₁₋₆-alkyl, C₂₋₆-alkenyl or C₂₋₆-alkynyl; wherein:
The term “C₁₋₆-alkyl” refers to a branched or unbranched alkyl group having from one to six carbon atoms, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl, pent-3-yl, hex-1-yl, hex-2-yl and hex-3-yl;
The term “C₂₋₆-alkenyl” refers to a branched or unbranched alkenyl group having from two to six carbon atoms and one double bond, including but not limited to ethenyl, propenyl, and butenyl; and
The term “C₂₋₆-alkynyl” refers to a branched or unbranched alkynyl group having from two to six carbon atoms and one triple bond, including but not limited to ethynyl, propynyl and butynyl.

The term “2′ O-Methyl RNA” or “2′ O-MeRNA” as used herein refers to a modified RNA nucleotide, wherein a methyl group is added to the 2′ hydroxyl of the ribose moiety of the nucleoside, producing a methoxy group. 2′ O-MeRNA nucleotides can hybridise with DNA or RNA according to Watson-Crick base-pairing rules and results in increased stability of the double-stranded oligonucleotide.

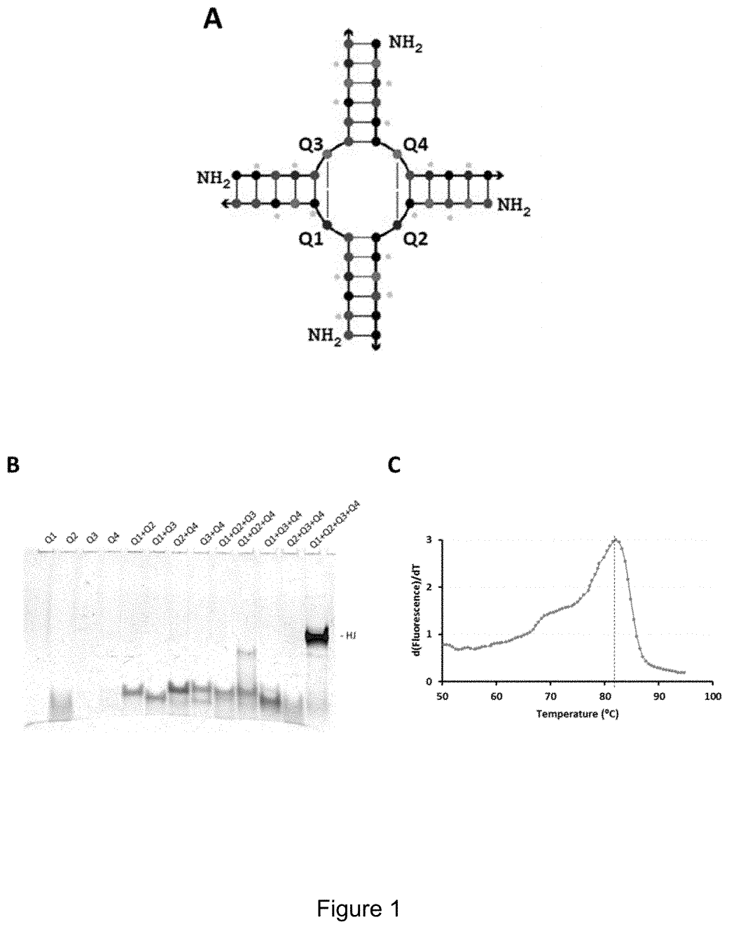

The term “double-stranded arm” as used herein refers to a “branch” or an arm wherein two strands have hybridised thereby generating said arm or branch. A nanodevice comprising three double-stranded arms are illustrated in FIG. 1A.

The term “amine” as used herein includes primary, secondary and tertiary amines such as —NH₂, —NH(R′) and —N(R′)₂, but also quartenary ammonium ion of the type —N⁺(R′)₃, guanidinium, imidazole, indole, pyridine or pyridinium. R represents an alkyl or other organic substituent.

The term “amide” as used herein includes primary, secondary and tertiary amides such as RC(O)NH₂, RC(O)NHR′ and RC(O)NR′R″, but also phosphoramides such as R₂P(O)NH₂, R₂P(O)NHR′ and R₂P(O)NR′R″, and sulfonamides such as RS(O)₂NH₂, RS(O)₂NHR′ and RS(O)₂NR′R″. R represents an alkyl or other organic substituent.

The term “thiol” as used herein is an organosulfur compound that contains a carbon-bonded sulfhydryl group (—C—SH) or sulphydryl group (R—SH), where R represents an alkyl or other organic substituent.

The term “carboxylates” as used herein includes salts of a carboxylic acid having the formula M(RCOO)n, where M is a metal and n is an integer such as 1, 2, 3, . . . , and esters of a carboxylic acid having the formula RCOOR′. R represents an alkyl or other organic substituent.

The term “haloacetyl” as used herein includes compunds having the formula XCH₂(O)R, wherein X represents halogen such as Cl, Br, I and F and R represents an alkyl or other organic substituent.

The term “azide” as used herein is an anion with the formula N³.

The term “aldehyde” as used herein is a compound having the formula —CHO.

The terms “Phosphorothioate-DNA” and “Phosphorothioate-RNA” refers a DNA analogue and an RNA analogue, respectively, wherein one of the non-bridging oxygens in the internucleotide linkage is replaced by sulphur.

The terms “morpholino-DNA” and “morpholino-RNA” s a type of oligomer molecule (colloquially, an oligo) used in molecular biology to modify gene expression. Its molecular structure has DNA/RNA bases attached to a backbone of methylenemorpholine rings linked through phosphorodiamidate groups. Morpholinos block access of other molecules to small (˜25 base) specific sequences of the base-pairing surfaces of ribonucleic acid (RNA). Nanodevice

One aspect of the present invention relates to nanodevice comprising of a branched nucleic acid structure comprising at least three double-stranded arms, wherein at least two of said double-stranded arms comprise:
LNA and/or modified LNA; and
at least one component selected from the group consisting of DNA, Phosphorothioate-DNA, morpholino-DNA, 2′-O-MeRNA, 2′-F-RNA, 2′MOE-RNA, 2′-O-Me-Phosphorothioate-RNA, 2′-F-Phosphorothioate-RNA, 2′MOE-Phosphorothioate-RNA, 2′-O-morpholino-MeRNA, 2′-F-morpholino-RNA and 2′MOE-morpholino-RNA,

wherein at least one of said double-stranded arms is modified with at least one functional chemical group, at least one linker and/or at least one ligand.

In one embodiment thereof the double-stranded arms comprise:
LNA and/or modified LNA; and
at least one component selected from the group consisting of DNA, 2′-O-MeRNA, 2′-F-RNA and 2′MOE-RNA.

A preferred embodiment of the present invetion relates to a nanodevice comprising of a branched nucleic acid structure comprising at least three double-stranded arms, wherein at least two of said double-stranded arms comprise LNA and 2′-OMe-RNA and wherein at least one of said double-stranded arms is modified with at least one functional chemical group, at least one linker and/or at least one ligand.

The nanodevice of the present invention comprises at least three nucleotide strands that are hybridised thereby generating a branched nucleic acid structure comprising at least three double-stranded arms. For example, three nucleotide strands are used to make a nucleic acid structure having three double-stranded arms, whereas four nucleotide strands are used to make a nucleic acid structure having four double-stranded arms. If for example four nucleotide strands are used, strands 1 and 2 may each hybridise to strand 3 and strand 4 such that approximately half of the nucleotides of each strand hybridises to half of the nucleotides of another strand thereby generating a nucleic acid structure having four double-stranded arms. It is not required that each arm of the nanodevice have the same length. Thus, each double-stranded arm may comprise a different number of nucleotides. An embodiment of a nanodevice comprising four double-stranded arms is illustrated in FIG. 1.

In one embodiment the nanodevice comprises at least three nucleotide strands or preferably at least four nucleotide strands. In a preferred embodiment the nanodevice comprises 3 nucleotide strands. In another preferred embodiment the nanodevice comprises four nucleotide strands.

The nanodevice may comprise from 3 to 6 double-stranded arms, preferably from 3 to 5 arms. In a more preferred embodiment the nanodevice comprises 3 or 4 double-stranded arms. Preferably the nanodevice comprises 4 double-stranded arms.

The nanodevice of the present invention comprises LNA and 2′-OMe-RNA in at least two double-stranded arms. In a preferred embodiment the nanodevice comprises LNA and 2′-OMe-RNA in at least three double-stranded arms, in a more preferred embodiment the nanodevice comprises LNA and 2′-OMe-RNA in at least four double-stranded arms.

In one embodiment the nanodevice comprises three double-stranded arms, wherein at least two of said arms comprise LNA and 2′-O-MeRNA. In a particular embodiment thereof, the nanodevice comprises LNA and 2′-OMe-RNA in two arms. In a more preferred embodiment the nanodevice comprises three double-stranded arms, wherein each arm comprises LNA and 2′-O-MeRNA.

In another embodiment the nanodevice comprises four double-stranded arms, wherein at least two of said arms comprise LNA and 2′-O-MeRNA. In a particular embodiment thereof, the nanodevice comprises LNA and 2′-OMe-RNA in two arms or more preferably in three arms. In a more preferred embodiment the nanodevice comprises four double-stranded arms, wherein each arm comprises LNA and 2′-O-MeRNA.

In another embodiment the nanodevice comprises five double-stranded arms, wherein at least two of said arms comprise LNA and 2′-O-MeRNA. In a particular embodiment thereof, the nanodevice comprises LNA and 2′-OMe-RNA in two arms or more preferably in three arms or even more preferably in four arms. In a most preferred embodiment thereof the nanodevice comprises five double-stranded arms, wherein each arm comprises LNA and 2′-O-MeRNA.

In a particular embodiment thereof, the nanodevice comprises LNA and 2′-OMe-RNA in two arms. In a more preferred embodiment the nanodevice comprises three double-stranded arms, wherein each arm comprises LNA and 2′-O-MeRNA.

In another embodiment the nanodevice comprises four double-stranded arms, wherein at least two of said arms comprise LNA and 2′-O-MeRNA. In a particular embodiment thereof, the nanodevice comprises LNA and 2′-OMe-RNA in two arms or more preferably in three arms. In a more preferred embodiment the nanodevice comprises four double-stranded arms, wherein each arm comprises LNA and 2′-O-MeRNA.

Each nucleotide strand of the nanodevice forms part of two double-stranded arms. Thus, in one embodiment at least one nucleotide strand comprises LNA and 2′-OMe-RNA resulting in a nanodevice, wherein at least two double-stranded arms comprise LNA and 2′-O-MeRNA.

In a preferred embodiment at least one nucleotide strand comprise LNA and 2′-O-MeRNA. In a more preferred embodiment at least three nucleotide strands comprise LNA and 2′-O-MeRNA. In an even more preferred embodiment at least four nucleotide strands comprise LNA and 2′-O-MeRNA.

In another embodiment at least one nucleotide strand comprises LNA and at least two nucleotide strands comprise or consist of 2′-OMe-RNA resulting in a nanodevice, wherein at least two double-stranded arms comprise LNA and 2′-O-MeRNA. In another embodiment at least one nucleotide strand comprises or consists of 2′-OMe-RNA and at least two nucleotide strands comprises LNA.

In one embodiment the nanodevice comprises three double-stranded arms, wherein one nucleotide strand comprises LNA and two nucleotide strands comprise or consist of 2′-O-MeRNA. In another embodiment the nanodevice comprises three double-stranded arms, wherein one nucleotide strand comprises or consist of 2′-OMe-RNA and two nucleotide strands comprise LNA.

In a preferred embodiment the nanodevice of the present invention comprises four double-stranded arms, wherein at least one nucleotide strand comprises LNA and at least two nucleotide strands comprise or consist of 2′-O-MeRNA. In another embodiment the nanodevice comprises three four stranded arms, wherein at least one nucleotide strand comprises or consist of 2′-OMe-RNA and at least two nucleotide strands comprise LNA. In one embodiment thereof, one nucleotide strand comprises LNA and two or three nucleotide strands comprise or consist of 2′-O-MeRNA. In a particular embodiment, two nucleotide strands comprise LNA and two nucleotide strands comprise or consist of 2′-O-MeRNA.

It is preferred that at least 50% of the nucleotides of the nanodevice is LNA and/or 2′-O-MeRNA. In a more preferred embodiment at least 60%, such as at least 70%, such as for example at least 80% or such as at least 90% of the nucleotides of the nanodevice is LNA and/or 2′-O-MeRNA. In one embodiment 100% of the nucleotides of the nanodevice are LNA and/or 2′-O-MeRNA.

The nucleotide strands that form the nanodevice preferably have a length of from 6 to 20 nucleotides. In another embodiment the nucleotides have a length of from 8 to 20 nucleotides, such as from 8 to 18 nucleotides, such as for example from 8 to 16 nucleotides, such as from 10 to 16 nucleotides or more preferably from 10 to 14 nucleotides.

In one embodiment the nucleotides have a length of 6, 7, 8 or 9 nucleotides or more preferably 10, 11 or 12 nucleotides. The nucleotides may also have a length of 13, 14, 15, 16, 17, 18, 19 or 20 nucleotides.

The nucleotide strands that form the nanodevice of the present invention may have different lengths. Thus, the length of each nucleotide of the nanodevice is not necessary the same.

As described above, each nucleotide strand of the nanodevice hybridises with two other strands to form the nanodevice. The double-stranded arms may for example have a length of from 3 to 10 base pairs. In another embodiment the double-stranded arms have a length of from 4 to 10 base pairs, such as from 4 to 9 base pairs, such as for example from 4 to 8 base pairs, such as from 5 to 8 base pairs or more preferably from 5 to 7 base pairs.

In one embodiment the double-stranded arms have a length of 3 or 4 base pairs or more preferably 5, 6 or 7 base pairs. The double-stranded arms may also have a length of 8, 9 or 10 nucleotides.

The double-stranded arms may have different lengths. Thus, the length of each double-stranded arms of the nanodevice is not necessary the same.

The nanodevice according to the present invention comprises at least two double-stranded arms comprising LNA and 2′-O-MeRNA. Thus, the at least two arms each comprise at least one LNA and at least one 2′-O-MeRNA. The at least two double-stranded arms do not necessarily comprise the same amount of LNA and 2′-O-MeRNA. The at least two double-stranded arms may for example comprise at least 2, at least 3, such as at least 4, at least 5 or at least 6 LNAs. In another embodiment at least three such as four double-stranded arms comprise at least 2, at least 3, such as at least 4, at least 5 or at least 6 LNAs.

In another embodiment the at least two double-stranded arms comprise at least 2 LNAs and at least one 2′-O-MeRNA, such as at least three 2′-O-MeRNA, at least 4 2′-O-MeRNA, at least 5 2′-O-MeRNA, at least 6 2′-O-MeRNA, at least 7 2′-OMe-RNA or at least 8 2′-O-MeRNA.

In yet another embodiment the at least two double-stranded arms comprise at least 3 LNAs and at least one 2′-O-MeRNA, such as at least three 2′-O-MeRNA, at least 4 2′-O-MeRNA, at least 5 2′-O-MeRNA, at least 6 2′-OMe-RNA or at least 7 2′-O-MeRNA.

In another embodiment the at least two double-stranded arms comprise at least 4 LNAs and at least one 2′-O-MeRNA, such as at least three 2′-O-MeRNA, at least 4 2′-O-MeRNA, at least 5 2′-OMe-RNA or at least 6 2′-O-MeRNA.

In a preferred embodiment at least three double-stranded arms comprise at least 2 LNAs and at least one 2′-O-MeRNA, such as at least three 2′-O-MeRNA, at least 4 2′-O-MeRNA, at least 5 2′-O-MeRNA, at least 6 2′-O-MeRNA, at least 7 2′-OMe-RNA or at least 8 2′-O-MeRNA.

In another preferred embodiment at least three double-stranded arms comprise at least 3 LNAs and at least one 2′-O-MeRNA, such as at least three 2′-O-MeRNA, at least 4 2′-O-MeRNA, at least 5 2′-O-MeRNA, at least 6 2′-OMe-RNA or at least 7 2′-O-MeRNA.

In yet another preferred embodiment at least three double-stranded arms comprise at least 4 LNAs and at least one 2′-O-MeRNA, such as at least three 2′-O-MeRNA, at least 4 2′-O-MeRNA, at least 5 2′-OMe-RNA or at least 6 2′-O-MeRNA.

In a preferred embodiment at least four double-stranded arms comprise at least 2 LNAs and at least one 2′-O-MeRNA, such as at least three 2′-O-MeRNA, at least 4 2′-O-MeRNA, at least 5 2′-O-MeRNA, at least 6 2′-O-MeRNA, at least 7 2′-OMe-RNA or at least 8 2′-O-MeRNA.

In another preferred embodiment at least four three double-stranded arms comprise at least 3 LNAs and at least one 2′-O-MeRNA, such as at least three 2′-O-MeRNA, at least 4 2′-O-MeRNA, at least 5 2′-O-MeRNA, at least 6 2′-OMe-RNA or at least 7 2′-O-MeRNA.

In yet another preferred embodiment at least four double-stranded arms comprise at least 4 LNAs and at least one 2′-O-MeRNA, such as at least three 2′-O-MeRNA, at least 4 2′-O-MeRNA, at least 5 2′-OMe-RNA or at least 6 2′-O-MeRNA.

In particular embodiment the nanodevice of the present invention comprises four double-stranded arms, wherein each arm comprises at least 2 LNAs and at least one 2′-O-MeRNA, such as at least three 2′-O-MeRNA, at least 4 2′-O-MeRNA, at least 5 2′-O-MeRNA, at least 6 2′-O-MeRNA, at least 7 2′-OMe-RNA or at least 8 2′-O-MeRNA.

In another particular embodiment the nanodevice of the present invention comprises four double-stranded arms, wherein each arm comprises at least 3 LNAs and at least one 2′-O-MeRNA, such as at least three 2′-O-MeRNA, at least 4 2′-O-MeRNA, at least 5 2′-O-MeRNA, at least 6 2′-OMe-RNA or at least 7 2′-O-MeRNA.

In yet another particular embodiment the nanodevice of the present invention comprises four double-stranded arms, wherein each arm comprises at least 4 LNAs and at least one 2′-O-MeRNA, such as at least three 2′-O-MeRNA, at least 4 2′-O-MeRNA, at least 5 2′-OMe-RNA or at least 6 2′-O-MeRNA.

It is preferred that most of the nucleotides of the nanodevice are LNA or 2′-O-MeRNA.

The functional chemical group, linker and/or ligand can be attached to the 5′ end and/or the 3′ end of the nucleotide strand. Preferable, the functional chemical group, linker and/or ligand is/are attached to an LNA and/or a 2′-O-MeRNA.

The functional chemical group, linker and/or ligand can also be attached to modified LNA, such as 2′-amino-LNA. Thus, the nanodevice of the present invention may comprises at least one 2′-amino-LNA. Accordingly, in one embodiment the functional chemical group, linker and/or ligand is/are attached to the amino-group of said 2′-amino-LNA.

In one embodiment the nanodevice comprises at least one ligand and further comprises at least one functional chemical group and/or at least one linker. In this embodiment, the ligand can be attached to the nanodevice via the functional chemical group or the linker. Thus, in one embodiment the ligand is attached to the functional chemical group or the linker. The linker and the functional chemical group may then be directly attached to the nanodevice in accordance with the embodiments described above.

In another embodiment, the ligand is attached to the linker and the linker is attached to the functional chemical group. In this embodiment, the functional chemical group is preferably directly attached to the nanodevice.

In yet another embodiment the ligand is attached to the functional chemical group and the functional chemical group is attached to the linker. In this embodiment, the linker is preferably directly attached to the nanodevice.

Ligand

The nanodevice may comprise at least one ligand, such as at least two ligands, such as at least three ligands or such as at least four ligands. One specific embodiment relates to a nanodevice comprising four double-stranded arm and at least two ligand, at least three ligand or at least four ligands. As described above, the ligands can be attached either directly to the nanodevice or via the functional chemical group and/or the linker.

The ligand(s) may for example be selected from the group consisting of therapeutic agents, imaging agents, targeting agents, aptamers, vitamins, antibodies, peptides, proteins, oligonucleotides, fluorophores, lipids, tags, small molecules and reactive chemical groups.

It is preferred that the nanodevice of the present invention is non-immunogenic, when not comprising a ligand that induces an immune response. Thus, the nanodevice may comprise a ligand that initiates an immune response, thereby resulting in a nanodevice that is immunogenic.

Thus, in one embodiment the ligand is a protein, such as for example an antibody or a nanobody. The antibody may for example be an antibody that recognises cancer cells.

The protein may also be an immune activating agent such as for example an antigen or an epitope, for example a tumour associated antigen or epitope. The immune activating agent may also be a peptide comprising a CpG motif.

In another embodiment the ligand is a targeting agent such as for example an agonist or an antagonist that binds to a specific receptor such as for example the LHRH receptor, Somatostatin receptor, Transferrin receptor or Insulin receptor. In one embodiment the targeting agent is a tumour targeting agent, such as for example a protein or a peptide comprising the motif Arg-Gly-Asp (RGD) and/or Asn-Gly-Arg (NGR).

In one embodiment the targeting agent is TriGalactoseamine (TriGalNAc). TriGalNAc ligand for the hepatocyte asialoglycoprotein receptor (ASGP-R). The nanodevice may for example comprise at least one TriGaINAc. Preferably the nanodevice comprises at least two TriGalNAc. Thus, the nanodevice may comprise 1 TriGaINAc, preferably 2 TriGalNAc, 3 TriGalNAc or 4 TriGalNAc. In a preferred embodiment thereof the nanodevice comprises 4 double-stranded arms. As shown in the example section, TriGalNAc can be used as a targeting agent for specific targeting of for example drugs to the liver.

Peptides containing the NGR and/or RGD motifs are known to bind CD13 isoforms expressed in tumor vessels and can be used for e.g. tumor targeting and targeted drug delivery.

In another embodiment the ligand is an aptamer. An aptamer is an oligonucleotide that may bind to a specific target, such as for example prostate specific membrane antigen (PSMA), Burkitt's lymphoma Transferrin receptor, Insulin receptor vascular cell adhesion molecule 1 (VCAM-1) or CD133 antigen also known as prominin-1.

In one embodiment the aptamer is used as a targeting agent. The aptamer may for example be an aptamer which binds to the prostate specific membrane antigen (PSMA) were (see example section). Thus, the aptamer can be used for specific targeting to PSMA-positive prostate cancer cells (LNCaP). In one embodiment the nanodevice comprises an aptamer and PEG. The nanodevice may for example comprise 1, 2, 3 or 4 aptamers.

In yet another embodiment the ligand is a nucleic acid such as for example an antisense oligo, small interfering RNA (siRNA), or micro RNA (miRNA).

In another embodiment the ligand is a small molecule. The small molecule may for example be a sugar such as galactose, galactoseamine, mannose, glucose or fructose.

In a further embodiment the ligand is an imaging agent such as fluorophore such as for example Cy3, Cy5, Cy5.5, Cy7 or Cy7.5.

In a further embodiment the ligand is an imaging agent such as for example green fluorescent protein (GFP), red fluorescent protein (RFP), yellow fluorescent protein (YFP) or luciferase.

The ligand may also be a peptide such as for example a cell-penetrating peptide. Cell-penetrating peptide may for example include blood-brain barrier-permeable peptide dNP2, Trans-Activator of Transcription (TAT), R9 (Poly Lysine), penetratin or angiopep.

The ligand may also be a chemical reactive group such as for example dibenzocyclooctyne (DBCO), alkyne, azide, maleimide, thiol or phosphine.

In one embodiment the ligand is a chelating agent, such as for example 1,4,7,10-tetraazacyclododecane-1,4,7,10-tetraacetic acid (DOTA) or 1,4,7-triazacyclononane-N,N′,N″-triacetic acid (NOTA).

In another embodiment the ligand is a pharmacokinetic (PK) enhancer. A pharmacokinetic enhancer is used to improve or boost the effectiveness of another drug. The PK enhancer may for example be albumin or a protein comprising or consisting of an Fc domain.

The ligand can also be a lipid such as cholesterol or fatty acid. In one embodiment the ligand is a vitamin such as biotin or folate. Thus, in a specific embodiment the nanodevice comprises at least 1 folate or preferably at least 2 folates. The nanodevice may for example comprise 1 folate, preferably 2 folates, 3 folates or 4 folates. In a preferred embodiment thereof the nanodevice comprises 4 double-stranded arms and 1 folate, preferably 2 folates, 3 folates or 4 folates. In one embodiment thereof, the nanodevice further comprises a fluorophore such as for example Cy3, Cy5, Cy5.5, Cy7 or Cy7.5.

For example the ligand is a tag, such as streptavidin, a histidine tag or a FLAG tag.

In a further embodiment the ligand is a cytostatic agent, such as for example a tubulin inhibitor. The ligand may also be a drug.

In one preferred embodiment the nanodevice of the ligand is Polyethylene glycol (PEG). As shown in the examples below, PEG can extend the lifetime of the nanodevice.

Functional Chemical Group

The nanodevice may comprise at least one functional chemical group, such as at least two functional chemical groups, such as at least three functional chemical groups or such as at least four functional chemical groups. As described above, the functional chemical group may for example be attached directly to the nanodevice and subsequently used the attached a linker or a ligand to the nanodevice.

In one embodiment said functional chemical group is selected from the group consisting of amines, amides, thiols, phosphates, carboxylates, haloacetyls, azides and aldehydes.

Detectable Moiety

The nanodevice may comprise in one preferred embodiment one or more detectable moieties. The detectable moiety can be unique for specific nanodevices, thus allowing the detection and/or quantification of each nanodevice in a pool of multiple nanodevices. This can be used for multiplex analyses.

The moiety may be radioactive tracer, a fluorescent probe or a unique oligonucleotide.

In preferred embodiment, the detectable moiety is a molecular barcode, which is a unique oligonucleotide. The nucleic acid barcode sequence preferably comprises at least 5 nucleotides, preferably 5-10 nucleotides, such as 8 nucleotides.

Where the detectable moiety consists of a unique oligonucleotide, it is easily detectable using standard assays available in the field. For example unique oligonucleotides can be detected enzymatically by standard multiplex qPCR or DNA sequencing, or by DNA PAINT. However other methods are also available.

The detectable moiety may be coupled directly to the nanodevice or attached via a linker.

Linker

The nanodevice may comprise at least one linker, such as at least two linkers, such as at least three linkers or such as at least four linkers.

The linker may be used to couple the ligand to the nanodevice. The linker can be attached directly to the nanodevice or it can be attached via a functional chemical group which is bound directly to the nanodevice.

In one embodiment the linker is selected from the group consisting of N-Hydroxysuccinimide (NHS), maleimide, dibenzocyclooctyne (DBCO), azide, sulpho-NHS, thiol and aldehyde.

The linker may for example be selected from the group consisting of N-Hydroxysuccinimide, maleimide and dibenzocyclooctyne.

Medical Use

The nanodevice of the present invention can be used for drug delivery such as targeted drug delivery. Thus, in another aspect, the present invention relates to a nanodevice as described herein and above for use as a medicament. In this embodiment, the nanodevice comprises a drug or a compound for medical use. That is, the nanodevice is bound to or coupled with a drug or a compound for medical use.

The drug can be coupled to the nanodevice via a linker as described herein above. The nanodevice may further comprise a targeting agent. The targeting agent is used for targeted delivery of the drug.

Thus, in another aspect the present invention relates to use of a nanodevice as defined herein and above for drug delivery. Preferably, said drug delivery is targeted drug delivery.

In another embodiment the nanodevice comprises two different drugs, three different drugs or four different drugs. The nanodevice may also be used to deliver two or more drugs in a specific stoichiometric relationship, such as when it is desired to deliver two drugs in a molar ratio of for example 1:2.

Pharmaceutical Composition

As described above, the nanodevice of the present invention can be used as a medicament, wherein the nanodevice comprises a drug. Whilst it is possible for said nanodevice to be administered alone, it is preferred to present them in the form of a pharmaceutical formulation.

Thus, another aspect of the present invention relates to a pharmaceutical composition comprising the nanodevice as defined herein and above.

It is preferred that the pharmaceutical composition further comprises at least one pharmaceutically acceptable carrier. Suitable carriers and the formulation of such pharmaceuticals are known to a person skilled in the art.

The pharmaceutically acceptable carriers can be either solid or liquid. Solid form preparations include powders, tablets, pills, capsules, cachets, suppositories, and dispersible granules. A solid carrier can be one or more excipients which may also act as diluents, flavoring agents, solubilizers, lubricants, suspending agents, binders, preservatives, wetting agents, tablet disintegrating agents, or an encapsulating material.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The nanodevice of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Pharmaceutically acceptable salts of the ligands or drugs coupled to the nanodevice, where they can be prepared, are also intended to be covered by this invention. These salts will be ones which are acceptable in their application to a pharmaceutical use. By that it is meant that the salt will retain the biological activity of the parent drug and the salt will not have untoward or deleterious effects in its application and use in treating diseases.

The nanodevice of the present invention may be formulated in a wide variety of formulations for parenteral administration.

For injections and infusions the formulations may take such forms as suspensions, solutions, or emulsions in oily or aqueous vehicles, for example solutions in aqueous polyethylene glycol. Alternatively, the active ingredient may be in powder form, obtained by aseptic isolation of sterile solid or by lyophilisation from solution for constitution before use with a suitable vehicle, e.g., sterile, pyrogen-free water. The formulations can be presented in unit-dose or multi-dose sealed containers, such as ampoules, vials, pre-filled syringes, infusion bags, or can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Examples of oily or non-aqueous carriers, diluents, solvents or vehicles include propylene glycol, polyethylene glycol, vegetable oils, and injectable organic esters, and may contain formulatory agents such as preserving, wetting, emulsifying or suspending, stabilizing and/or dispersing agents.

The compounds of the present invention may be formulated in a wide variety of formulations for oral administration. Solid form preparations may include powders, tablets, drops, capsules, cachets, lozenges, and dispersible granules. Other forms suitable for oral administration may include liquid form preparations including emulsions, syrups, elixirs, aqueous solutions, aqueous suspensions, or solid form preparations which are intended to be converted shortly before use to liquid form preparations, such as solutions, suspensions, and emulsions.

In powders, the carrier is a finely divided solid which is a mixture with the finely divided active component. In tablets, the active component is mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. Suitable solvents for the preparation of an oily solution include glycerol, diluted alcohol and propylene glycol.

Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well-known suspending agents.

In one embodiment the pharmaceutical composition comprises an additional active agent.

Administration Forms

As described herein above administration forms include but are not limited to oral, parental, enteral, rectal or buccal administration.

In one embodiment the pharmaceutical composition is administered or adapted for administration enterally, parenterally or as part of a sustained release implant. The parenteral administration may for example be intravenous, subcutaneous, intramuscular, intracranial or intraperitoneal.

In a preferred embodiment the pharmaceutical composition is administered by or adapted for injection, such as parenteral injections. Other drug-administration methods, such as subcutaneous injection, which is effective to deliver the drug to a target site or to introduce the drug into the bloodstream, are also contemplated.

Methods and Use

A further aspect of the present invention relates to a method of treating, preventing or ameliorating a disease by administering to a subject in need thereof a therapeutically effective amount of the nanodevice as described herein and above.

The compound may also be administered in the form of a pharmaceutical composition as described herein and above. Administrations forms are as described herein and above.

Generally, “a subject in need thereof” is an individual, who suffers from a specific disease or disorder.

The subject may be any animal or human. In a preferred embodiment the subject is a human.

Another aspect of the present invention relates to use of a nanodevice as defined herein for bioimaging. In this embodiment the nanodevice comprises an imaging agent such as a bioimaging agent.

In one embodiment the imaging agent is a fluorophore such as for example Cy3, Cy5, Cy5.5, Cy7 or Cy7.5. In another embodiment the imaging agent is selected from the group consisting of green fluorescent protein (GFP), red fluorescent protein (RFP), yellow fluorescent protein (YFP) or luciferase.

In another aspect, a method is provided for screening nanodevices with specific functionalities. Thus, in one aspect, a method is provided of selecting a nanodevice as defined herein, said method comprising

a. providing one or more of said nanodevices

b. subjecting said nanodevice to a functional test

c. selecting those nanodevices that respond successfully to said test.

The terms that the nanodevices “respond successfully to said test” is meant to imply that specific desired functional features are detected in response to a functional test. How and when a nanodevice is determined to respond successfully of course depends on the type and intention of the particular assay.

The functional test can be any test for the presence of any relevant functional feature of a nanodevice as described herein. For example, the test can be a cellular uptake test, where, the screening method is directed to identifying those nanodevices, which are able to accumulate within cells, or in a particular intracellular compartment. For example within specific cell types, in particular cancer cells. Thus, nanodevices, which are capable of being uptaken/accumulating within specific cells will in such assays be detected as responding successfully.

In another embodiment, the functional test is a ligand binding test, where nanodevices are selected on the basis of the ability to bind a specific ligand. Such an assay may test specific conditions for ligand binding, specificities of the ligand binding and binding efficiency. Particularly cellular targets can be interesting, such as extracellular proteins, and in particular cancer-specific ligands are of interest. Successful responders would include those nanodevices which are capable of binding the relevant ligand being tested.

The ability of nanodevices to cross a biological barrier is another example of a functional test, where nanodevices are selected on the basis of their ability to cross different biological barriers, in particular cell membranes or structures that mimic cell membranes in vitro. The test may also relate to the nanodevices' ability to accumulate in a specific tissue. Specifically, cancer tissues are contemplated but also other tissues or organs can be relevant. Successful responders would then include those nanodevices which are capable of crossing the biological barrier being tested.

Finally, the functional test can be directed to the nanodevices' ability to inhibit or accelerate a specific activity, for example an an enzymatic assay or the interaction of two reactants. In this case, successful responders are those nanodevices, which display an ability to inhibit or accelerate said enzymatic reaction. Another example could be the ability to prevent receptor dimerization

In a particularly preferred embodiment, each nanodevice comprises a unique detectable moiety. This will allow a multiplex analysis, where a mixture comprising a plurality of different nanodevices each comprising a unique detectable moiety are subjected to a functional test, followed by a selection of those nanodevices that respond successfully to said test, and then identifying the specific nanodevices, which responds successfully to the test on the basis of their unique detectable moiety.

Examples of detectable moieties are described herein above.

### EXAMPLES

Synthesis of Oligonucleotides

All modified oligonucleotides were synthesized on an automated oligonucleotide synthesizer via standard phosphoramidite chemistry in 1 μmol scale. Commercially available 2′-OMe-RNA and LNA phosphoramidites were used in combination with the relevant modifiers (C6 amino modifier), linkers and spacers. Synthesized oligonucleotides were deprotected and cleaved from the solid support by standard deprotection conditions, and the crude oligonucleotides were purified by HPLC, and their composition verified by MALDI-TOF MS analysis. The sequences of all oligos used in this study are shown in Table 1.

TABLE 1
Oligonucleotides used in this study.

| | SEQ | |
|---|---|---|
| Name | ID NO: | Sequence* |
| Q1 | 1 | AminoC6 mCICmGITmCmCmTIGmAIGmCmC |
| Q2 | 2 | AminoC6 mCmAICmAIGmTmGIGmAICmGmG |
| Q3 | 3 | AminoC6 mGIGmCITmCmAmCmCIGmAITmC |
| Q4 | 4 | AminoC6 mGmAITmCIGmGmAmCITmGITmG |
| Q3_PSMA1_ | 5 | tagaattctaatacgactcactataGGGCTCA |
| Templ_F | | CCGATCGGGAGGACGATGCGGA |
| Q3_PSMA1_ | 6 | TTAGGAGTGACGTAAACATGGCTGATCCGCAT |
| Templ_R | | CGTCCTCCCGATCGGTGAGCCC |
| Q3_PSMA2_ | 7 | tagaattctaatacgactcactataGGGAGGA |
| Templ_F | | CGATGCGGATCAGCCATGTTTA |
| Q3_PSMA2_ | 8 | GATCGGTGAGCCTTAGGAGTGACGTAAACATG |
| Templ_R | | GCTGATCCGCATCGTCCTCCCT |

*Unless otherwise specified all nucleotides are deoxynucleotides. m = 2′OMe, f = 2′ F, I = LNA.

Bioconjugation

Each Holliday Junction (HJ) module contains a primary amine in the 5′end allowing us to perform highly specific bioconjugation relying on NHS esters. Whenever possible, commercially available NHS esters to attach specific functional molecules in a single step were employed. In those cases where NHS esters were not available or not compatible with the inherent chemistry of the ligands, a two-step conjugation reaction utilizing a number of heterobifunctional linkers were used. All molecules used, as well as their sources are listed in Tables 2 and 3.

For the functional molecules listed in Table 2, a 5′-amine coupled oligo was reacted with a commercially available NHS ester using the following general protocol: 1, 10 or 50 nmol oligo was prepared in NHS reaction buffer (50 mM HEPES, pH 8.2, 30% DMSO) at a final concentration of 0.5 mM. To this was added from different stock solutions 5-50 fold molar excess of the NHS ester. For NHS esters that were insoluble in water (such as NHS-DBCO) the DMSO concentration was adjusted to 50%. Reactions were stopped after 4 hours by ethanol precipitation. The resulting conjugation reactions were dissolved in 100 μl RNase free water and purified by RP-HPLC using a C18 column. Fractions containing the appropriate conjugates were collected, quantified by UV spectroscopy and subsequently freeze dried for long term storage. All conjugates were verified by gel electrophoresis.

TABLE 2
List of NHS-coupled ligands used in this study

| Name | Description/Reference |
|---|---|
| NHS-PEG20K | Nanocs (PG1-SC-20k) |
| NHS-PEG10K | Nanocs (PG1-SC-10k) |
| NHS-PEG5K | Nanocs (PG1-SC-5k) |
| NHS-Cy3 | Lumiprobe (#41020) |
| NHS-Cy5 | Lumiprobe (#43020) |
| NHS-Cy5.5 | Lumiprobe (#47020) |
| NHS-Cy7 | Lumiprobe (#45020) |
| NHS-Cy7.5 | Lumiprobe (#46020) |
| NHS-DBCO | Lumiprobe (#54720) |
| NHS-Maleimide | Thermo (#22322) |
| NHS-DOTA | Macrycyclics |
| NHS-Azide | BaseClick (BCL-014-5) |
| NHS-folate | BaseClick (BCFA-111-5) |
| NHS-Biotin | Jena Bioscience (CLK-B103-25) |

Ligands containing azides or free thiols (Table 3) were reacted with DBCO- or maleimide-coupled oligos in a 3:1 (ligand to oligo) ratio in DBCO reaction buffer (50 mM HEPES, pH 7.5) at a concentration of at least 100 μM. The final conjugates were purified by RP-HPLC followed by quantification and freeze drying as described above.

Assembly and Characterization of Holliday Junction

HJ oligos were stored at −80° C. in stock solutions of 1 mM, 100 μM and 10 μM in RNase free water. Equimolar amounts of each oligo (Q1-4) were mixed at a final concentration of 10-100 μM in one of the following buffers: assembly buffer (200 mM KOAc, pH 7.5), Phosphate buffered saline (PBS, pH 7.4) or TAEM (40 mM tris, pH 8.3, 1 mM EDTA, 12 mM MgCl₂). The mixtures were incubated at room temperature for approximately 30 min. In order to study the assembly kinetics, oligos were pre-incubated in pure water precluding the oligos from forming intermolecular basepairings. At specific timepoints, concentrated buffer was added allowing complex formation to initiate. The assembly process could then be directly visualized by gel electrophoresis.

TABLE 3
List of Azido- or thiol-
coupled ligands used in this study

| | SEQ | |
|---|---|---|
| | ID | |
| Name | NO: | Description/Reference |
| TriGalactoseamine | | This study |
| AngioPep2 | 9 | Azp-TFFYGGSRGKRNNFKTEEY- |
| | | OH. Biosynthan |
| Penetratin | 10 | Azp-RQIKIWFQNRRMKWKK-OH. |
| | | Biosynthan |
| THR | 11 | Azp-THRPPMWSPVWP-OH. |
| | | Biosynthan |
| aSynuclein | | Full length human aSyn |
| | | with a C-terminal |
| | | Cysteine. Daniel Otzen, |
| | | iNANO |

In Vitro Transcription

2-F RNA aptamers were prepared with a 5′ or 3′ overhang replacing Q3 in the HJ structure (see Table 1). Template DNA carrying a T7 promoter site were annealed and transcribed essentially as described previously^{[4]}. Correct sized RNAs were excised from 12% polyacrylamide gels and purified by phenol/chloroform extraction followed by ethanol precipitation. The aptamers incorporation into the HJ was analyzed by gel electrophoresis.

Cell Lines and In Vitro Uptake Studies

HepG2 cells were grown in EMEM medium (ATTC), PC3 cells were grown in F12 Kaighn's Modification medium (GE Healthcare), KB cells were grown in RPMI-1640 medium (Sigma-Aldrich) and LNCaP cells were grown in RPMI-1640 medium (ATTC), all supplemented with 10% fetal bovine serum (FBS) (Gibco) and 1% penicillin/streptomycin (P/S) (full medium), at 37° Celsius (C), 5% CO₂. Prior to seeding, the cells were washed with PBS (Dulbecco) and released by incubating with 0.05% Trypsin-EDTA (1×, Gibco) for 5-10 minutes (min.) Cells were centrifuged at 200 g for 5 min. and resuspended in full medium. Cell number and viability was determined by use of a Via 1-Casette counting chamber (Chemometer), using the software NucleoView NC-200.

Row Cytometry

For uptake studies, the following general protocol was used: Cells were seeded in full medium as 100,000-150,000 cells per well in a 24 well plate one day prior to treatment. Cells were incubated with medium for negative controls, and Cy5-labeled HJs for 30-45 min. in concentrations of 50 nanomolar (nM), 100 nM and 200 nM. Cells were washed three times with PBS and trypsinated for 10 min, re-suspended in full medium and centrifuged for 10 min at 1000 g. The supernatant was removed and the cells washed with PBS, centrifuged and resuspended in 300 microlitre (μL) PBS and transferred to flow cytometry tubes. Before seeding of LNCaP cells, a 24 well plate was coated with poly-L-lysine (PLL) at a concentration of 100 μg/mL in PBS) for 15 min. at room temperature, followed by washing with PBS. Flow cytometry was performed on a Gallios flow cytometer (Beckman Coulter) and analysed using Kaluza software.

Confocal Microscopy

An 8 well microscopy plate was coated with PLL (100 μg/mL) for 15 min. at room temperature, followed by washing with PBS. 150,000 LNCaP cells were seeded out in full medium one day prior to treatment. Samples were added to the medium of the cells (medium, Cy5-labeled HJ or HJ-PSMA in 200 nM) and incubated at 37° C. for 45 min. The medium was removed, and the cells were washed three times with PBS. The cells were stained with 200 μL 2.5 μg/mL wheat germ agglutinin (WGA)-Alexa 488 for 15 min. at 37° C. The cells were washed three times with PBS and fixed by adding 200 μL 4% paraformaldehyde (PFA) and incubated at 37° C. for 15 min. Samples were washed with PBS, dried and stained with one drop of DAPI ProLong Gold (Invitrogen) to each position on the slide. After mounting of the cover slide, the sample was incubated overnight (ON) at 4° C. Cells were imaged on a confocal laser scanning microscope (Zeiss LSM 700) with a 63× oil objective.

Immunogenicity

Human monocytes and macrophages were isolated as peripheral mononuclear blood cells (PMBCs) from a buffy coat from a healthy donor, using Ficoll-Paque density gradient separation. The isolated collection of leukocytes were diluted in RPMI medium with endotoxin-free FBS and seeded out as 100,000 cells per well in a round-bottom 96 well plate. Four hours after seeding, cells were treated with lipoplexes containing the different variations of HJs in three replicates and incubated for 18 hours at 37° C. The cells were centrifuged, and cell-free supernatant was collected and diluted with ELISA diluent buffer. The samples were subsequently added to a coated ELISA plate along with a serial titration of recombinant TNF-α standard for the calculation of a standard curve. From this standard curve, the amount of TNF-α measured in the different samples was calculated.

Animal Experiments

Studies were performed on 10-week old adult female BALB/c mice. Animals had free access to a standard rodent diet and water. During the experiments, animals were kept in groups of 4-5 mice per cage.

In Vivo Pharmacokinetics and Biodistribution in Mice

For pharmacokinetic studies of PEGylated HJs using gel electrophoresis, female BALB/c mice were injected with approximately 500 picomole (pmol) of Cy5.5-labeled Q3, HJ and HJ-PEG20-60K through tail vein injection (n>5 for each construct). Blood samples were taken from the tail at different time points over 24 hours using Microvette 300 tubes (Sarstedt). The blood serum was collected by centrifugation of the tubes for 5 min. at 10,000 g. 10 μL serum was mixed with 50 μl PBS and subsequently scanned in a 96-well black Illumino plate (Thermo Scientific). For subcutaneous injections (S.C.) 500 pmol samples in PBS were injected above the tail.

All scanning was performed on an IVIS 200 instrument (Xenogen, Caliper Life Sciences, Hopkinton, Mass., USA) using Living image 4.3 software (Caliper Life Science). Fluorescent signals were subjected to spectral unmixing using the systems in-build Cy5.5 filter settings and an untreated mouse control. Individual blood samples were normalized to the injected total fluorescence, which was assumed from the 2 min. timepoint. At the end of the study, animals were sacrificed by cervical dislocation. The animal organs were removed and analysed as described above.

Liver Targeting

HJs containing 0, 1 or 2 TriGalactoseamine (TriGalNAc) ligand for the hepatocyte asialoglycoprotein receptor (ASGP-R) along with a Cy5.5 label were assembled as described above and 500 pmol samples injected intravenously (I.V). For time course experiments, the IVIS scanner was adjusted to take images every 2 min. over a period of 30 min. Prior to injection, the mice were shaved to better visualize liver uptake. For normal biodistribution studies, animals were sacrificed after 2 hours or 24 hours.

Tumor Targeting

LNCaP cells were grown as described above to approximately 70-80% confluence, washed and resuspended in PBS at a concentration of approximately 5×10⁷ cells/mL and subsequently mixed with an equal volume of Matrigel. The cells (200 μl) were injected S.C. into each flank of BALB/c male nude mice (8-weeks old) (BALB/c-AnNRj-Foxn1^{nu}) under isoflurane anaesthesia. Tumor growth was monitored every other day. After 30 days, tumor-bearing mice were separated in three groups: negative controls (n=2), Group 1 (n=5) and Group 2 (n=5). Each group was injected I.V. as described above with PBS (negative control), 3 nmol HJ-PEG2OK (Group 1) or 3 nmol HJ-PEG20K-A10 (Group 2). After 24 h, the animals were sacrificed and the organs scanned as described above.

### Example 1

Synthesis and Initial Characterization of the Holliday Junction Scaffold

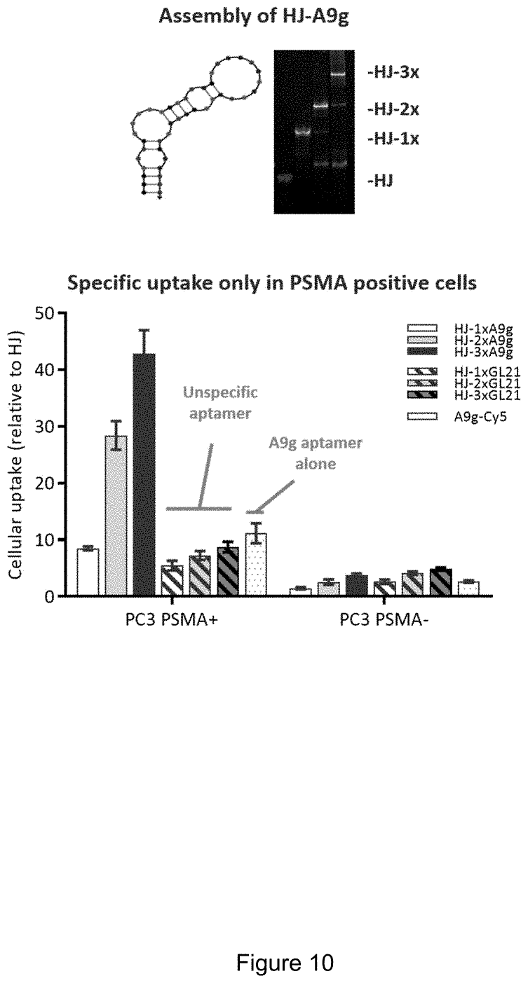

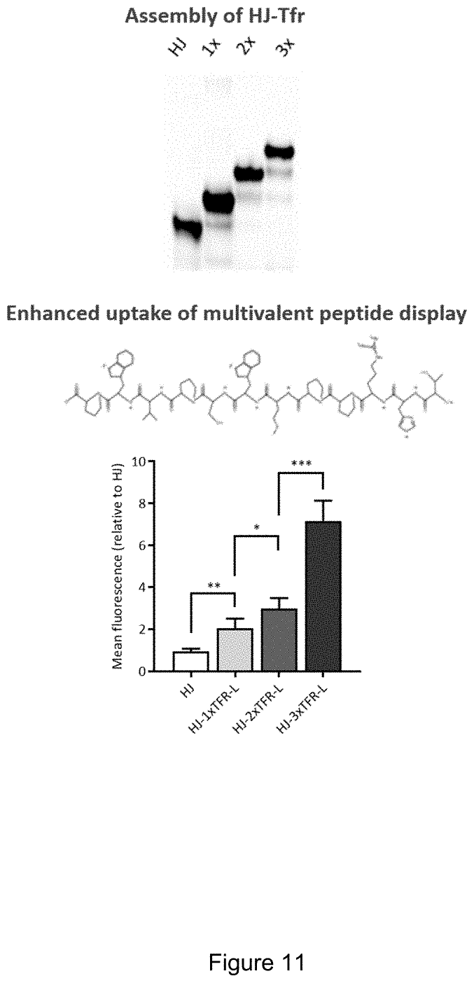

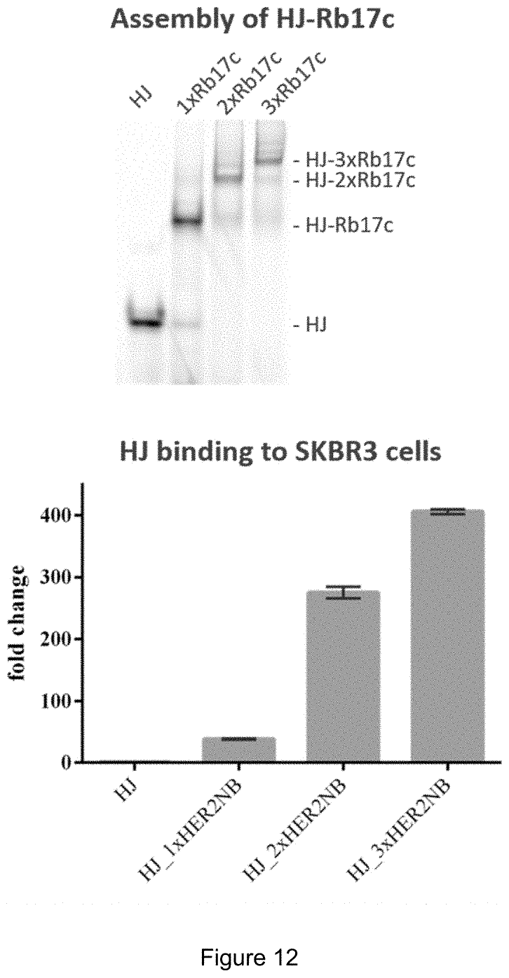

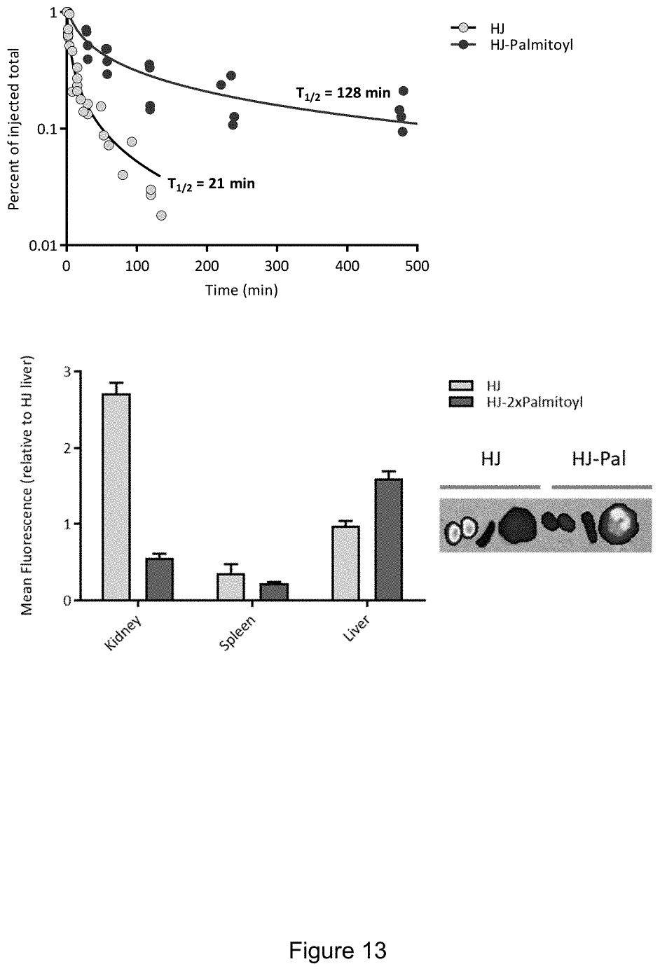

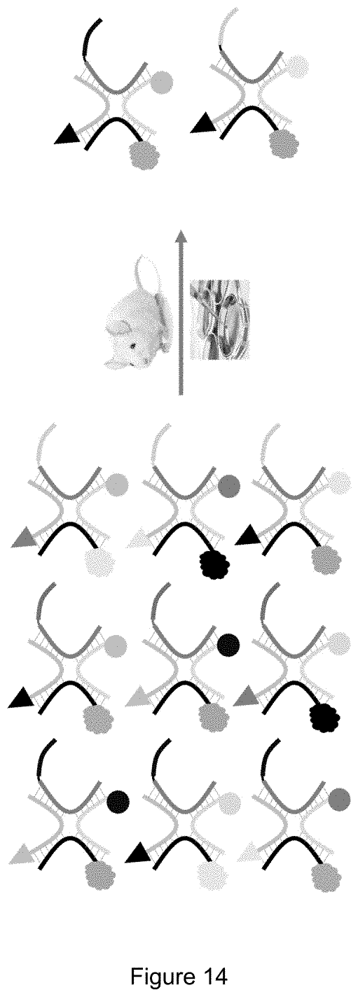

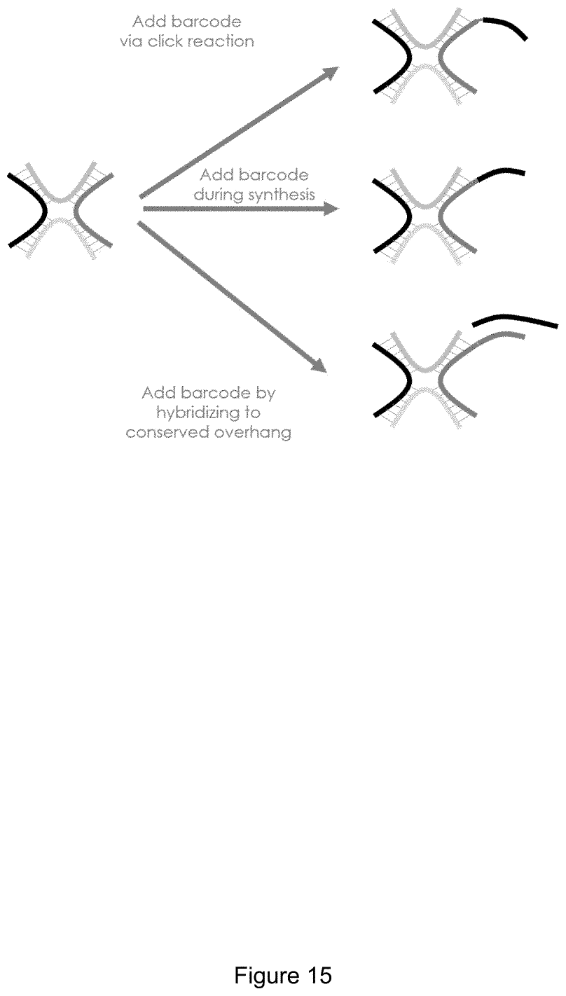

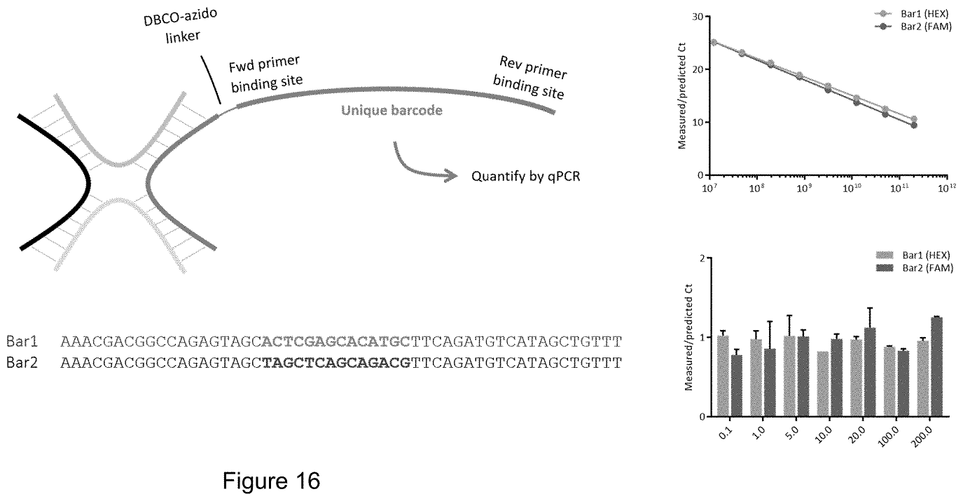

FIG. 1A shows a schematic illustration of a structure comprising four double-stranded arms and a mixture of 2′-OMe RNA and LNA nucleosides. The structure is also referred to as a Holliday Junction (HJ) structure or scaffold. The structure is an example of the device according to the present invention. Each of the double-stranded arms consists of six basepairs stabilized by four LNAs. As shown in FIG. 1B, the HJ structure showed robust assembly with no unspecific interactions and was extremely stable. The predicted Tₘ for a 24 nucleotide duplex stabilized by 16 LNAs is 85° C. (https://eu.idtdna.com/calc/analyser). In contrast, the predicted Tₘ for a corresponding unmodified duplex is only 67° C. Using SYBRGold stain to measure the amount of double-stranded HJ, we measured the Tₘ of the intact Holliday junction at 82.3° C. (FIG. 10).

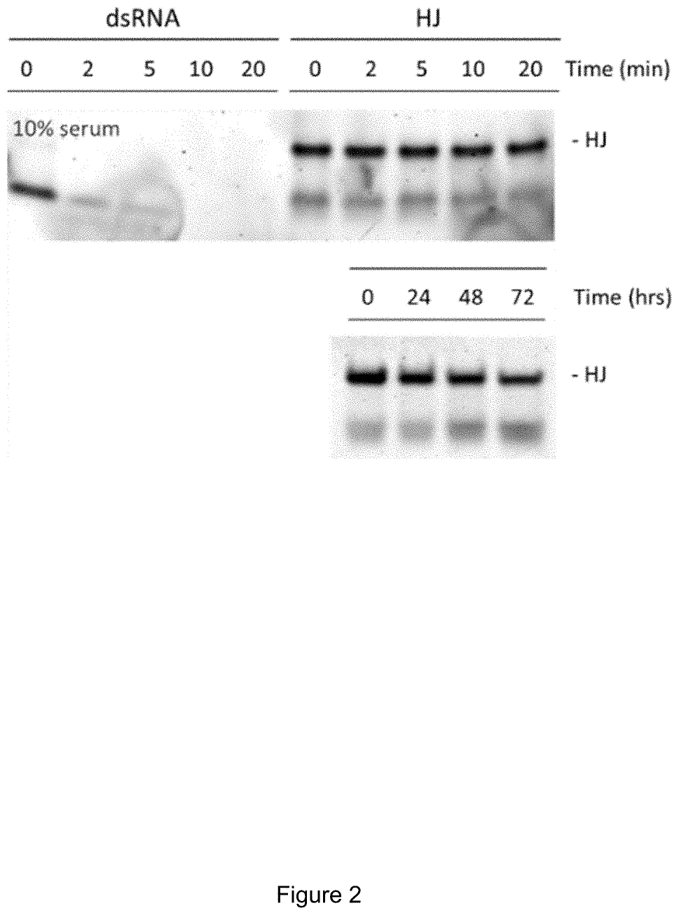

Since the HJ scaffold was intended for use in biological systems, the stability of the assembled structure were tested over time in fetal bovine serum (FBC). As shown in FIG. 2, The HJ scaffold remained surprisingly stable for several days in FBS while unmodified double-stranded RNA was degraded almost immediately.

### Example 2

Assembly of Functionalized Holliday Junctions

Each of the four strands of the HJ scaffold contains a 5′-amine group enabling straightforward conjugation to a number of commercially available functionalities. We attached a number of ligands to each of the four strands ranging from fluorophores (Cy3, Cy5, Cy5.5, Cy7, Cy7.5) to peptides (insulin, THR, Penetratin) to polyethylene glycol chains to small molecules (biotin, triGalNAc, folate). Although the total yields varied between different ligands we routinely recovered >50% of the initial oligonucleotide as a covalent oligo-ligand conjugate.

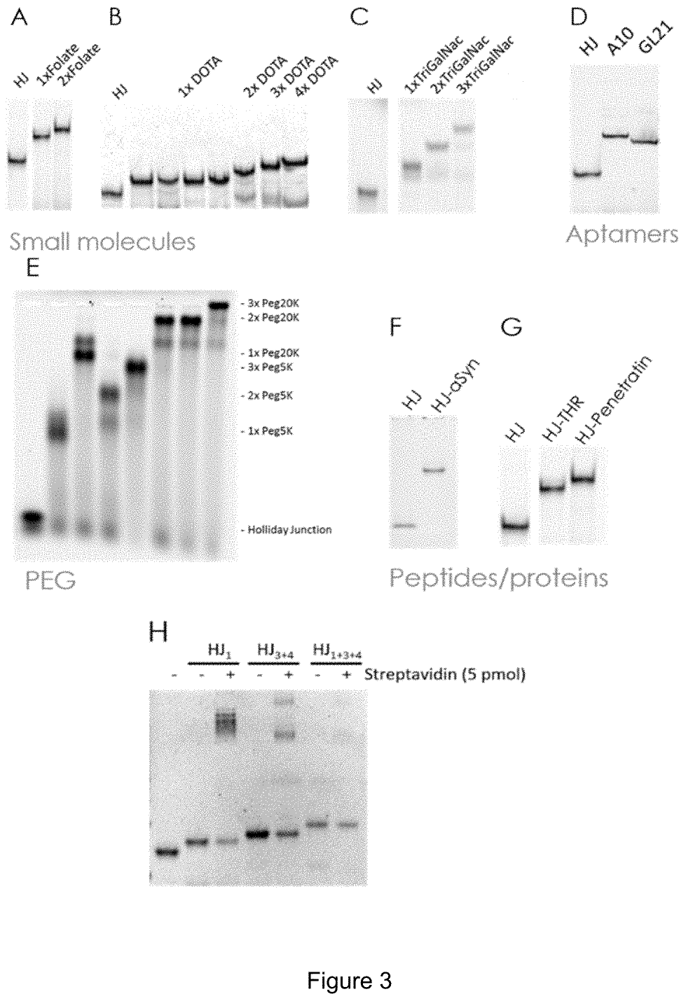

In order to measure the effect of each conjugate on the assembly of intact HJ, we used non-denaturing gel electrophoresis. In all cases tested, the functionalized Holliday junctions assembled without complications when mixed together in stoichiometric amounts at room temperature (FIG. 3).

### Example 3

Immunogenicity

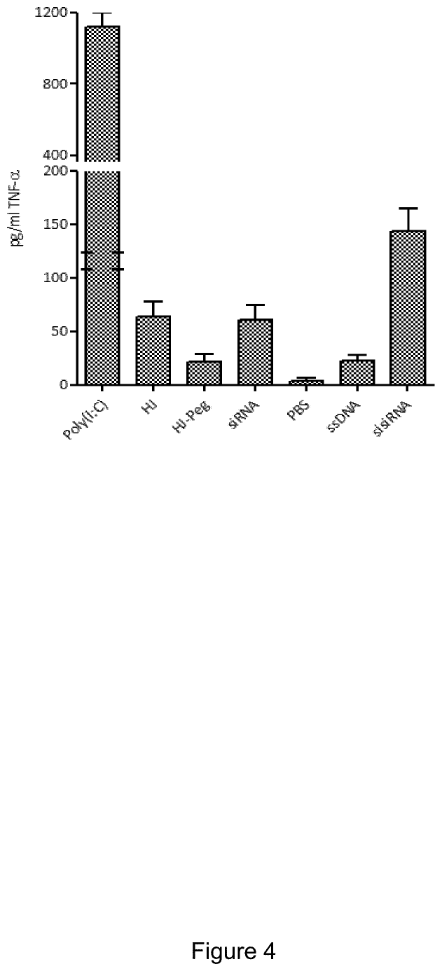

In order to assess the immunogenicity of the HJ, peripheral mononuclear blood cells (PMBCs) isolated from a buffy coat from a healthy donor were used to measure the induction of tumour necrosis factor alpha (TNF-α) in response to the addition of a number of different nucleic acids. The results are presented in FIG. 4 and show that while the addition of polyIC resulted in a significant induction of TNF-α, the addition of HJ led only to a small increase in TNF-α levels. Importantly, this increase was not significantly different from that observed following the addition of siRNAs and could be significantly reduced by including a PEG module in the HJ structure.

### Example 4

Biodistribution of PEGylated HJs

In order to use the HJ as a theranostic device, it is important to understand its behavior inside the body.

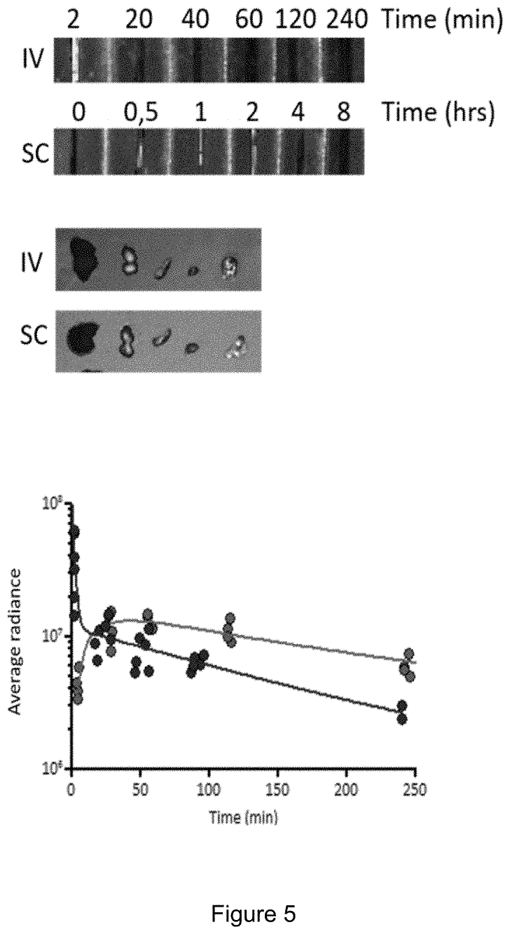

Cy5.5 labeled HJs were injected either I.V. or S.C. and measured blood levels over 24 hrs. I.V. injected HJs disappeared quickly from the blood and the majority of the fluorescent signal was subsequently found in the kidney (FIG. 5) suggesting that the Hjs are removed by renal clearance. The S.C. injected HJs reached their maximum blood levels after approximately 2 hrs consistent with a slow release rate into the blood stream. However, after 24 hrs the biodistribution profile was identical to that of the I.V. injected mice.

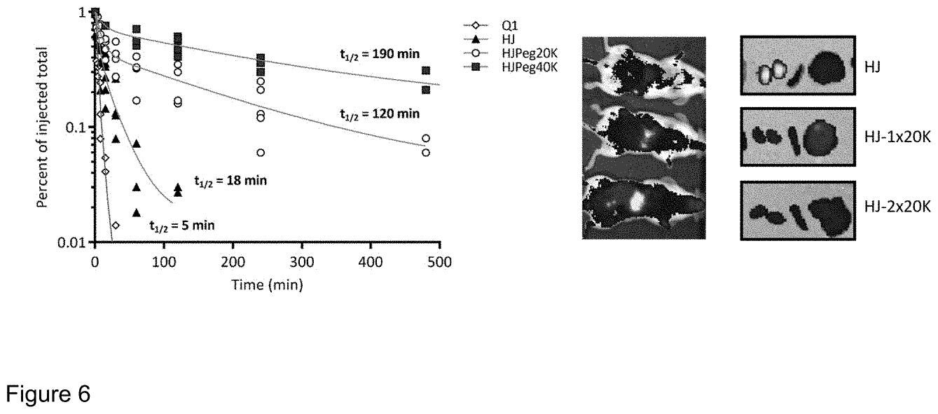

In order to increase the circulation time of the HJs, we attached 20 kDa PEG chains to one or more of the HJ arms. As before, non-conjugated HJs and free Cy5.5 labeled oligos were rapidly cleared from the blood stream (FIG. 6). In contrast, HJ functionalized with a single 20 kDa PEG chain continues to circulate with an apparent half-life of approximately 120 minutes. The addition of two PEG20K chains resulted in HJs that could still be detected in the blood even after 24 hrs. The addition of three PEG20K chains did not increase circulation time significantly (data not shown).

### Example 5

Targeted Delivery In Vitro

To investigate cell- and tissue-specific uptake of the HJs, we attached the small molecule targeting ligand, folate, to two of the HJ oligos and assembled HJs with one or two folates and a Cy5 fluorophore. We incubated KB cells expressing high levels of the folate receptor with different amounts of HJ. Following extensive washing, cellular uptake of Cy5-labeld HJs was quantified by flow cytometry. Subsequent analysis by confocal microscopy showed that HJs were indeed present in the cytosol and not on the cell membrane (data not shown).

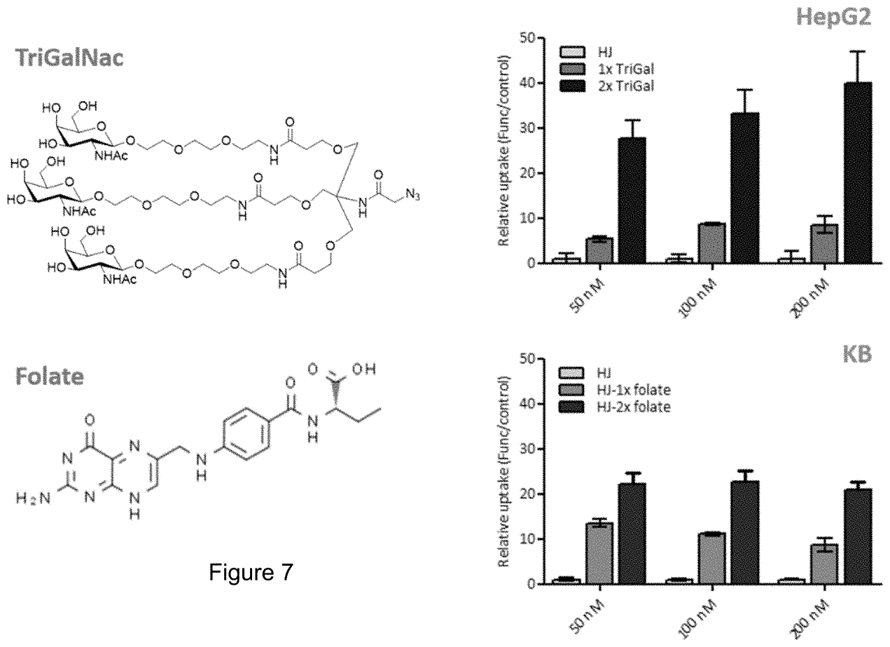

The results are presented in FIG. 7 and shows low background uptake for HJs without any ligands. The addition of a single folate resulted in a more than 8 fold higher uptake at the highest concentration. Attaching two folates increased cell uptake even further showing a 20-fold increase over free HJ at the highest concentration.

A “click” version of the triGalNAc, which could be used to functionalize one or more of the HJ arms, was synthesized. Similar to the folate experiment, the uptake of HJs with either one or two modifications on human heptocarcinoma cells (HepG2) was tested. The results are shown in FIG. 7. HJs functionalized with one triGalNAc showed a >8 fold increased uptake compared to free HJs at the highest concentration. Functionalizing two strands of the HJ gave an increase in uptake of up to 40 fold compared to free HJs in the same concentration, which was higher than the expected double effect of the two modifications.

### Example 6

Targeted Delivery In Vivo

The highly successful in vitro assays using triGalNAc prompted us to test the applicability of the HJs for in vivo delivery. In our pilot experiment, we injected mice with Cy5.5-labeled HJs functionalized with one or two triGalNAcs. The animals were sacrificed 2 hrs. post injection and the organs collected and scanned. The results showed a significantly higher fluorescent signal in the livers of animals injected with triGalNAc functionalized HJs (data not shown). In fact we could hardly detect any signal coming from the kidney. When we measured blood levels of triGalNAc-HJs we could see that the HJs were cleared from the blood just as rapidly as the non-functionalized HJs (data not shown). Collectively, these data suggested to us that the triGalNAc uptake in the liver happened very quickly.

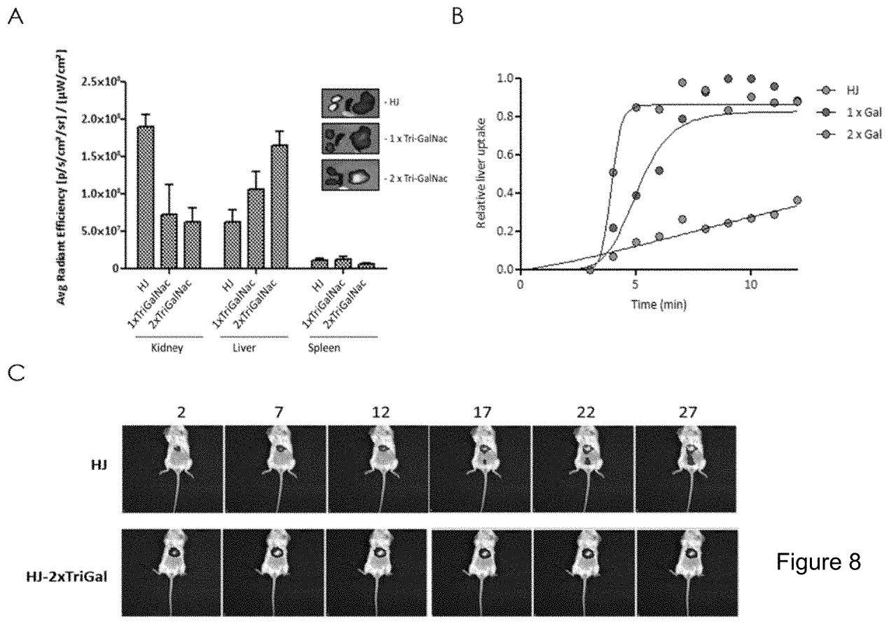

In order to measure the rapid uptake of triGalNAc functionalized HJ in the liver, the IVIS instrument was set to take images every 2 minutes. As shown in FIGS. 8B and 8C, the livers of animals injected with HJs carrying one or two triGalNAc were saturated almost immediately. In contrast, the livers from animals injected with non-functionalized HJs showed much slower uptake rates. Moreover, renal clearance was much more pronounced in these animals as evidenced by the emergence of a clear signal from the bladder after approximately 15 minutes (FIG. 8C).

Next a thorough biodistribution study using triGalNAc conjugated HJs was conducted. The results in FIG. 8A show that a single triGalNAc moiety is sufficient to reduce renal clearance. However, maximum liver targeting is achieved by the inclusion of an additional triGalNAc moiety.

### Example 7

Targeting Using Cancer Specific Aptamers

Aptamers present a number of advantages as targeting ligands compared to antibodies. When attaching aptamers to the device of the present invention a further advantage is obtained in that the aptamer can be synthesized with an unpaired overhang as one continuous strand. This overhang can then substitute for one of the HJ modules with the aptamer part protruding from one end. This eliminates the time consuming part of bioconjugation and purification.

An A10 aptamer which binds to the prostate specific membrane antigen (PSMA) were in vitro transcribed. Gel electrophoresis showed that the extended A10 did not compromise the self-assembly of the HJ (FIG. 3D). Moreover, Tm measurements of the construct showed a melting temperature comparable to the normal HJ (data not shown).

Next HJs were assembled with the aptamer and a Cy5-labeled Q2 strand for evaluating cellular uptake using flow cytometry. PSMA-positive prostate cancer cells (LNCaP) and PSMA-negative prostate cancer cells (PC3) were treated with either free or A10-functionalized HJs. Uptake efficiencies were measured by flow cytometry as before.

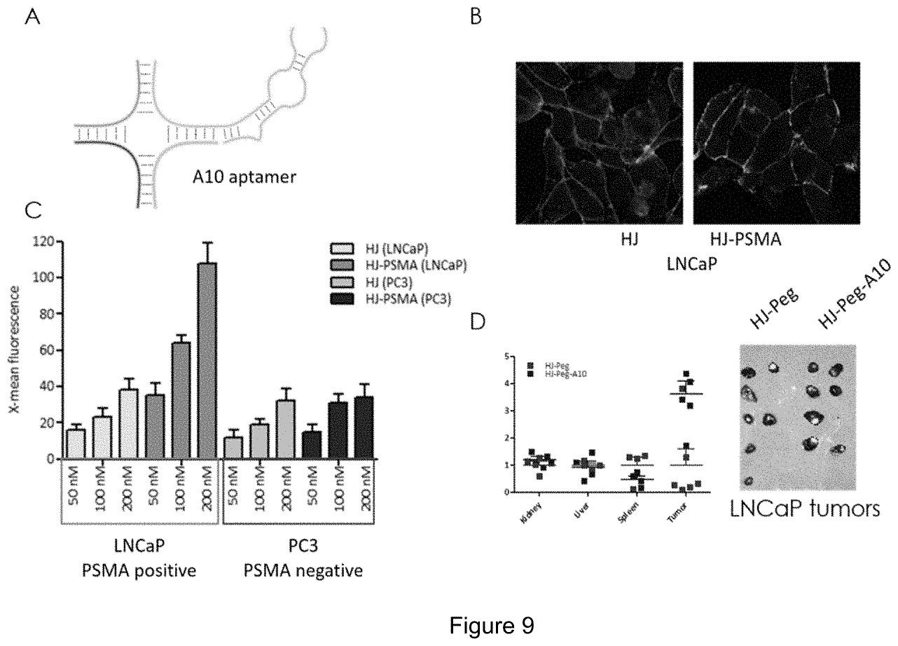

From the data presented in FIG. 9, it is seen that uptake of A10-elongated HJs in LNCaP cells was dose respondent and provided a more than 2.5 fold uptake compared to the background uptake of free HJ. This internalization appeared to be specific, as no increased uptake was observed in the PSMA-negative PC3 cells. Confocal microscopy imaging confirmed that the measured fluorscence signal in the LNCaP cells stems from the cell interior, indicating that the aptamer-functionalized HJs are internalized in these cells.

In order to study the ability of the A10 aptamer to direct HJs in vivo, we nude mice with grafted LNCaP tumors. The mice were injected with either HJs or HJs functionalized with the A10 aptamer. In order to increase circulation time to allow the aptamer sufficient time to associate with the tumor cells, we also included a 20 kDa PEG to both constructs. After 8 hrs, the animals were sacrificed and their organs and tumors scanned. The results are shown in FIG. 9D. In four of the five mice injected with non-functionalized HJs, we could not detect any fluorescent signal from the tumors. In contrast, a low signal was consistently observed in the tumors of all mice injected with the A10-HJ. Liver and kidney signals were not significantly different between the two groups. However, we did observe higher spleen signals from the non-functionalized HJ group (FIG. 9D, left part).

Discussion.

We have presented a novel nanodevice, which can be used as a platform for targeted delivery and bioimaging. The nanodevice at least three, such as for example four oligonucleotide strands that assemble rapidly at room temperature in a stoichiometric and quantitative fashion when mixed together. As shown herein, efficient protocols have been developed for attaching ligands, chemical groups and/or linkers to the nanodevice.

The nanodevice is remarkable stable (FIG. 2) and assembly of the nanodevice is highly specific and extremely robust.

The present invention provides a nanodevice that can be used for e.g. targeted delivery and bioimaging. The nanodevice is extremely versatile and in principle its use is only limited by the availability of suitable ligands.

Items

1. A nanodevice comprising of a branched nucleic acid structure comprising at least three double-stranded arms, wherein at least two of said double-stranded arms comprise:
LNA; and
at least one component selected from the group consisting of DNA, Phosphorothioate-DNA, morpholino-DNA, 2′-O-MeRNA, 2′-F-RNA, 2′MOE-RNA, 2′-O-Me-Phosphorothioate-RNA, 2′-F-Phosphorothioate-RNA, 2′MOE-Phosphorothioate-RNA, 2′-O-morpholino-MeRNA, 2′-F-morpholino-RNA and 2′MOE-morpholino-RNA, wherein at least one of said double-stranded arms is modified with at least one functional chemical group, at least one linker and/or at least one ligand.

2. The nanodevice according to item 1, wherein at least two of said double-stranded arms comprise LNA and 2′-OMe-RNA and wherein at least one of said double-stranded arms is modified with at least one functional chemical group, at least one linker and/or at least one ligand.

3. The nanodevice according to any of items 1 and 2, wherein said nanodevice comprises at least three nucleotide strands.

4. The nanodevice according to item 3, wherein said nanodevice comprises 3 to 6 double-stranded arms.

5. The nanodevice according to item 4, wherein said nanodevice comprises 3 to 5 double-stranded arms.

6. The nanodevice according item 5, wherein said nanodevice comprises four double-stranded arms.

7. The nanodevice according to any of the preceding items, wherein at least one of said nucleotide strands comprises LNA nucleotides and at least one of said nucleotide strands comprises of 2′-OMe-RNA nucleotides.

8. The nanodevice according to any of the preceding items, wherein said nucleotide strands have a length of from 6 to 20 nucleotides.

9. The nanodevice according to any of the preceding items, wherein said nanodevice comprises at least one 2′-amino-LNA.

10. The nanodevice according to item 9, wherein said functional chemical group, linker and/or ligand is attached to the amino-group of said 2′-amino-LNA.

11. The nanodevice according to any of the preceding items, wherein said nanodevice comprises at least one ligand and further comprises at least one functional chemical group and/or at least one linker.

12. The nanodevice according to item 12, wherein said ligand is attached to the functional chemical group or the linker.

13. The nanodevice according to item 13, wherein said ligand is attached to the linker and wherein the linker is attached to the functional chemical group.

14. The nanodevice according to any of the preceding items, wherein said nanodevice comprises at least two ligands, such as at least three ligands or such as at least four ligands.

15. The nanodevice according to item 15, comprising at least two different ligands, such as at least three different ligands or such as at least four different ligands.

16. The nanodevice according to any of items 12 and 14, wherein said ligand(s) is/are selected from the group consisting of therapeutic agents, imaging agents, targeting agents, aptamers, vitamins, antibodies, peptides, albumin, oligonucleotides, fluorophores, lipids, tags, small molecules and reactive chemical groups.

17. The nanodevice according to any of the preceding items, wherein said functional chemical group is selected from the group consisting of amines, amides, thiols, phosphates, carboxylates, haloacetyls, azides and aldehydes.

18. The nanodevice according to item 10, wherein said linker is selected from the group consisting of N-Hydroxysuccinimide, maleimide and dibenzocyclooctyne.

19. A nanodevice according to any of the preceding items for use as a medicament.

20. The nanodevice according to item 18, wherein said nanodevice comprises a drug.

21. A pharmaceutical composition comprising the nanodevice as defined in any of items 1 to 19.

22. The pharmaceutical composition according to item 20, further comprising a pharmaceutically acceptable carrier.

23. A method of treating, preventing or ameliorating a disease by administering to a subject in need thereof a therapeutically effective amount of the pharmaceutical composition according to item 21.

24. The method according to item 22, wherein said subject is a human.

25. Use of a nanodevice as defined in any of items 1 to 19 for bioimaging.

26. Use of a nanodevice as defined in any of items 1 to 19 for drug delivery.

27. The use according to item 21, wherein said drug delivery is targeted drug delivery.

1. A nanodevice comprising a branched nucleic acid structure comprising four double-stranded arms, wherein at least two of said double-stranded arms comprise:
LNA; and
2′-O-MeRNA,
wherein at least one of said double-stranded arms is modified with at least one functional chemical group, at least one linker and/or at least one ligand.

2. (canceled)

3. (canceled)

4. (canceled)

5. The nanodevice according to claim 1, wherein at least one of said nucleotide strands comprises LNA nucleotides and at least one of said nucleotide strands comprises of 2′-O-Me-RNA nucleotides.

6. The nanodevice according to claim 1, wherein said nucleotide strands have a length of from 6 to 20 nucleotides.

7. The nanodevice according to claim 1, wherein said nanodevice comprises at least one 2′-amino-LNA.

8. The nanodevice according to claim 1, wherein said functional chemical group, linker and/or ligand is attached to the amino-group of said 2′-amino-LNA.

9. The nanodevice according to claim 1, wherein said nanodevice comprises at least one ligand and further comprises at least one functional chemical group and/or at least one linker.

10. The nanodevice according to claim 9, wherein said ligand is attached to the functional chemical group or the linker.

11. The nanodevice according to claim 10, wherein said ligand is attached to the linker and wherein the linker is attached to the functional chemical group.

12. The nanodevice according to claim 1, wherein said nanodevice comprises at least two ligands.

13. The nanodevice according to claim 13, comprising at least two different ligands.

14. The nanodevice according to 9, wherein said ligand(s) is/are selected from the group consisting of therapeutic agents, imaging agents, targeting agents, aptamers, vitamins, antibodies, nanobodies, peptides, albumin, oligonucleotides, fluorophores, lipids, tags, small molecules and reactive chemical groups.

15. The nanodevice according to claim 1, wherein said functional chemical group an amines.

16. The nanodevice according to claim 1, wherein said linker is selected from the group consisting of N-Hydroxysuccinimide, maleimide and dibenzocyclooctyne.

17. (canceled)

18. (canceled)

19. The nanodevice according to claim 1 within a pharmaceutical composition.

20. The nanodevice according to claim 19, further comprising a pharmaceutically acceptable carrier.

21. A method of treating, preventing or ameliorating a disease by administering to a subject in need thereof a therapeutically effective amount of the pharmaceutical composition according to claim 20.

22. The method according to claim 21, wherein said subject is a human.

23. A method of selecting a nanodevice according to claim 1, said method comprising
a. providing one or more of said nanodevices
b. subjecting said nanodevice to a functional test
c. selecting those nanodevices that respond successfully to said test.

24. The method according to claim 23, wherein each nanodevice comprises a unique detectable moiety.

25. The method according to claim 23, wherein said functional test is a cellular uptake test, a ligand binding test, ability to cross a biological barrier test or ability to accumulate in a specific tissue or ability to survive a particular process.

26. (canceled)

27. (canceled)

28. (canceled)

29. The nanodevice according to claim 1, wherein said nucleotide strands have a length of from 10 to 14 nucleotides.

30. The nanodevice according to claim 1, wherein said functional chemical group, linker and/or ligand is attached to the 5′ end.


SEQUENCE LISTING (13 sequences)

SEQ ID NO: 1 - DNA, 12 bp, Artificial sequence
  Synthesized modified oligonucleotide
  misc_feature (1)..(12): Nucleotides 1,3, 5, 6, 7, 9, 11 and 12.are 2′-OMe-RNA. Nucleotides 2, 4, 8 and 10 are LNA (locked nucleic acid).
ccgtcctgagcc

SEQ ID NO: 2 - DNA, 12 bp, Artificial sequence
  Synthesized modified oligonucleotide
  misc_feature (1)..(12): Nucleotides 1, 2, 4, 6, 7, 9, 11 and 12 are 2′-OMe-RNA. Nucleotides 3, 5, 8 and 10 are LNA (locked nucleic acid).
cacagtggacgg

SEQ ID NO: 3 - DNA, 12 bp, Artificial sequence
  Synthesized modified oligonucleotide
  misc_feature (1)..(12): Nucleotides 1, 3, 5, 6, 7, 8, 10 and 12 are 2′-OMe-RNA. Nucleotides 2, 4, 9 and 11 are LNA (locked nucleic acid).
ggctcaccgatc

SEQ ID NO: 4 - DNA, 12 bp, Artificial sequence
  Synthesized modified oligonucleotide
  misc_feature (1)..(12): Nucleotides 1, 2, 4, 6, 7, 8, 10 and 12 are 2′-OMe-RNA. Nucleotides 3, 5, 9 and 11 are LNA (locked nucleic acid)
gatcggactgtg

SEQ ID NO: 5 - DNA, 54 bp, Artificial sequence
  Synthesized modified oligonucleotide
  misc_feature (1)..(54): All nucleotides are deoxynucleotides
tagaattctaatacgactcactatagggctcaccgatcgggaggacgatgcgga

SEQ ID NO: 6 - DNA, 54 bp, Artificial sequence
  Synthesized modified oligonucleotide
  misc_feature (1)..(54): All nucleotides are deoxynucleotides
ttaggagtgacgtaaacatggctgatccgcatcgtcctcccgatcggtgagccc

SEQ ID NO: 7 - DNA, 54 bp, Artificial sequence
  Synthesized modified oligonucleotide
  misc_feature (1)..(54): All nucleotides are deoxynucleotides.
tagaattctaatacgactcactatagggaggacgatgcggatcagccatgttta

SEQ ID NO: 8 - DNA, 54 bp, Artificial sequence
  Synthesized modified oligonucleotide
  misc_feature (1)..(54): All nucleotides are deoxynucleotides.
gatcggtgagccttaggagtgacgtaaacatggctgatccgcatcgtcctccct

SEQ ID NO: 9 - protein, 19 aa, Artificial sequence
  Azido- or thiol-coupled ligand
  MISC_FEATURE (1)..(19): AngioPep2
TFFYGGSRGKRNNFKTEEY

SEQ ID NO: 10 - protein, 16 aa, Artificial sequence
  Azido- or thiol-coupled ligand
  MISC_FEATURE (1)..(16): Penetratin
RQIKIWFQNRRMKWKK

SEQ ID NO: 11 - protein, 12 aa, Artificial sequence
  Azido- or thiol-coupled ligand
  MISC_FEATURE (1)..(12): THR
THRPPMWSPVWP

SEQ ID NO: 12 - DNA, 53 bp, Artificial sequence
  DNA barcode
  misc_feature (1)..(53): Example of a barcode system
aaacgacggccagagtagcactcgagcacatgcttcagatgtcatagctgttt

SEQ ID NO: 13 - DNA, 53 bp, Artificial sequence
  DNA barcode
  misc_feature (1)..(53): Example of a barcode system
aaacgacggccagagtagctagctcagcagacgttcagatgtcatagctgttt